(19) United States
(12) Patent Application Publication
(10) Pub. No.: US 2026/0090898 A1
(43) Pub. Date: Apr. 2, 2026

(54) Methods of Preparing a Femur for Insertion of a Prosthetic Femoral Stem Component

(71) Applicant: Midland Medical Technology Ltd, Clent (GB)

(72) Inventor: Derek James Wallace McMinn, Clent (GB)

(21) Appl. No.: 18/902,572

(22) Filed: Sep. 30, 2024

(51) Int. Cl. A61F 2/46; A61B 17/16; A61B 17/17; A61B 17/56

(52) U.S. Cl. CPC A61F 2/4607; A61B 17/1668; A61B 17/1703; A61B 17/175; A61B 2017/564; A61B 2217/005

(57) ABSTRACT

The disclosure relates to methods of preparing a femur for hemi arthroplasty or total hip replacement. An aim is to create a channel through the femur, for receipt of a vent tube, which can be attached to a surgical suction device to lower the pressure on the mid and distal femur and thereby prevent or at least reduce a risk of embolisation. The channel may be created from (and accessed via) a proximal end of the femur or a distal end of the femur. Furthermore, the methods disclosed herein may be selectively used with cemented or uncemented components and with flat (blade-shaped) or rounded (conical) femoral stem components.

### TECHNICAL FIELD OF THE DISCLOSURE

The disclosure relates to methods of preparing a femur for insertion of a prosthetic femoral stem component. Particularly but not exclusively, the methods are conceived for improved patient outcomes following hemi arthroplasty or total hip replacement.

### BACKGROUND OF THE DISCLOSURE

The present disclosure relates to methods for use in hip arthroplasty (also known as hip replacement). Hip arthroplasty includes total hip arthroplasty (THA) whereby an artificial femoral component including a head (i.e. ball) component is replaced along with an artificial acetabular cup (i.e. socket), and hemi-arthroplasty whereby only the femoral component is replaced and the natural acetabulum is retained.

In some cases, hip resurfacing may be performed instead of total hip arthroplasty or hemi-arthroplasty. In which case, only a damaged bone surface (e.g. of the head of the femur and the acetabular surface) is removed and replaced by prosthetic (head and cup) resurfacing components.

Information from clinical use of hip resurfacing (HR) implants compared to stemmed total hip arthroplasty (THA) has been published and has shown that the mortality of patients having stemmed total hip replacements is significantly higher at 6 to 10 years compared to hip resurfacing when all confounding factors are adjusted for.

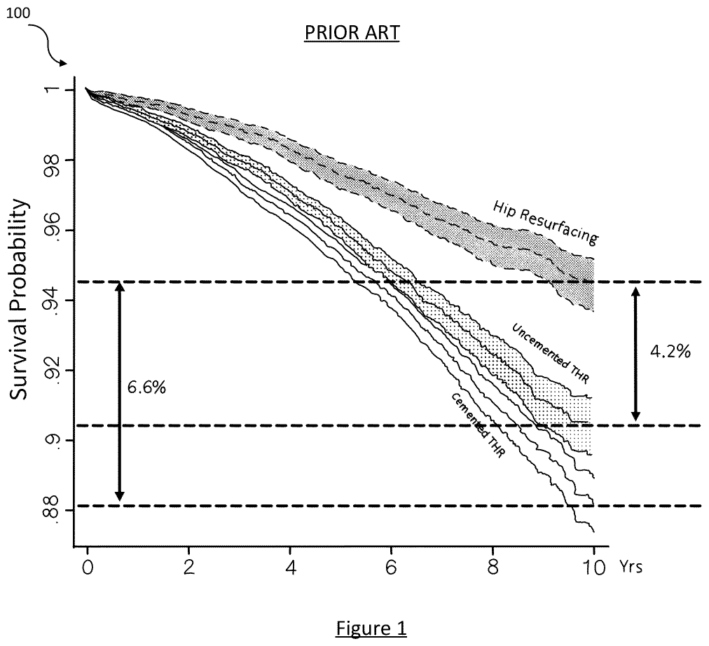

Three large studies each involving national level data have shown the same trend of mortality in the years following hip resurfacing being significantly lower than in the years following both un-cemented and cemented stem THA. It is futile to compare mortality in the years following a large group of HR patients to a large group of THA patients without adjusting for confounding factors. The main confounding factor is of course age at the time of surgery. A group of HR patients will have a lower mean age at surgery than a group of THA patients and with unadjusted data, this will give a false assessment of mortality. A key study to date with respect to adjusting for all confounding factors is entitled, “Mortality rates at 10 years after metal-on-metal hip resurfacing compared with THR in England; retrospective cohort analysis of hospital episode statistics”, BMJ 2013; 347 doi Kendall et al. November 2013. Using the English hospital episode statistics database linked to mortality records from the Office for National Statistics, the following confounding factors were adjusted for: age, sex, Charlson Comorbidity Index, rurality, area deprivation, surgical volume, and year of operation. FIG. 1 shows a graph 100 illustrating the results of this study. The graph 100 shows that 10 years after surgery, the survival probability of patients fitted with uncemented total hip replacement (THR) stems is 4.2% less than for hip resurfacing patients. Furthermore, the survival probability of patients fitted with cemented total hip replacement (THR) stems is 6.6% less than for hip resurfacing patients in these matched patient cohorts.

It is therefore an aim of the present disclosure to provide methods of preparing a femur for insertion of a prosthetic femoral stem component, with improved patient outcomes following hip arthroplasty.

### SUMMARY

Venting the femur in hip resurfacing, which was started by the Applicant and taught to surgeons across the world, has become the International norm. It is believed that it is the venting that accompanies hip resurfacing that results in low long-term mortality compared to THA, for which, at the time of writing, traditional techniques do not allow for successful venting of the femur.

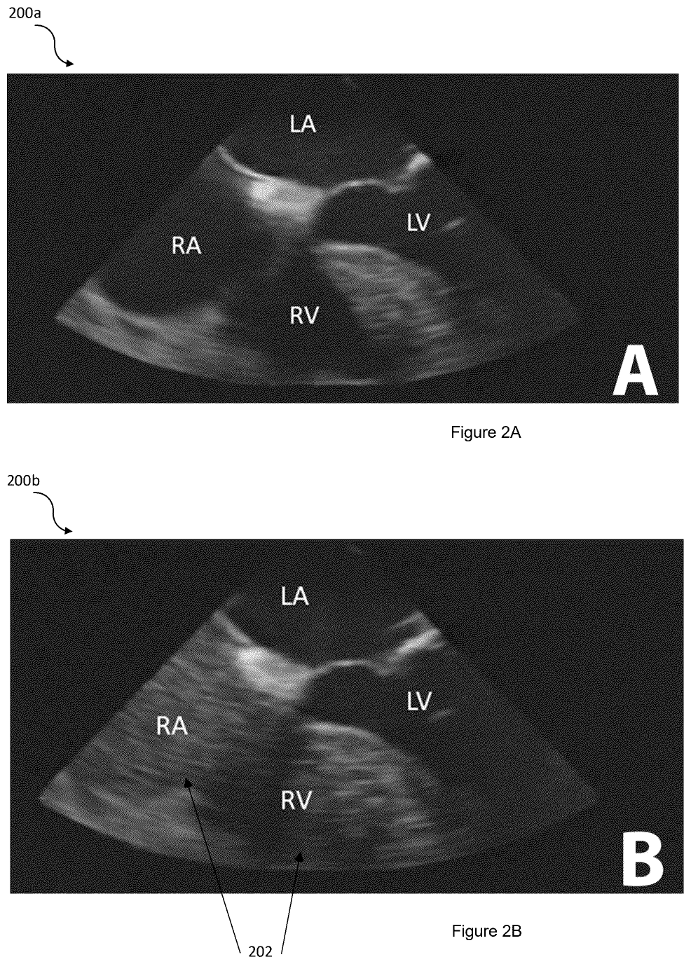

The present applicant investigated this phenomenon using echocardiograms and found that during THA surgery a massive amount of fat and marrow from the femur is displaced into the Right Atrium (RA) and Right Ventricle (RV) of the heart and this embolisation of debris can last for up to 20 minutes. On the contrary, HR patients show no embolisation on the echocardiogram. By way of illustration, FIG. 2A shows an echocardiogram 200a of a heart before any surgery to the femur and FIG. 2B shows a similar echocardiogram 200b following insertion of a cemented stem hip arthroplasty. The white features 202 in the Right Atrium (RA) and Right Ventricle (RV) of the echocardiogram 200b are fat and marrow. Blood from the Right Ventricle (RV) then is pumped into the lungs with oxygenated blood returning to the Left Atrium (LA) and Left Ventricle (LV), which is then pumped to all organs. It will be noted that there are no white features 202 in the Left Atrium (LA) and Left Ventricle (LV) of the echocardiogram 200b, indicating that the fat and marrow from the Right Atrium (RA) and Right Ventricle (RV) gets trapped in the lung microvascular bed.

The applicant believes that it is highly likely that this embolisation that accompanies insertion of stemmed hip arthroplasty is not good for the patient's wellbeing and life expectancy. However, 99% of all hip replacements are stemmed—so a major objective is to make stemmed hip replacements safer. To date, this embolisation during THA has not been successfully addressed.

When investigating the above, the applicant learned that the pressure generated in the femur during stemmed hip replacements is 1400 mm Hg (186651.31579 Pascals), which is roughly equivalent to car tyre pressure. The applicants therefore connected a car tyre to a cadaveric femur and discovered that all of the fat and marrow was evacuated through small vascular apertures in the femoral cortical bone. The applicant also discovered that a cement restrictor, typically placed below the tip of a cemented stem to prevent wet cement from entering the mid femoral shaft, had no effect of reducing the pressure build-up in the femoral canal below the cement restrictor.

In view of the above, the applicant believes that the increased mortality rate for stemmed hip procedures is a result of the plunger effect, which significantly raises the pressure in the distal femur and this causes embolisation of fat and marrow from the femur into veins and then to the Right Atrium (RA) and Right Ventricle (RV) of the heart, from where debris is pumped into the lungs and gets lodged in the micro-pulmonary vasculature.

Consequently, this disclosure proposes to overcome the above problems by providing methods of keeping intramedullary pressure low during insertion of uncemented and cemented total hip arthroplasty stems.

The applicant notes that HR patients have their femur vented by inserting a cannula through the lesser trochanter and into the femoral canal, during surgery. Since no embolisation is evident on echocardiograms for HR patients, the applicant proposes to vent the femur to prevent the dangerous rise in pressure during insertion of a THA stem, thereby preventing the increased mortality believed to result. In hip resurfacing patients, the femur is vented by applying strong suction through a cannula inserted via the lesser trochanter into the canal of the femur.

In general, the aim is to create a channel through the femur, for receipt of a vent tube, which can be attached to a strong surgical suction device to lower the pressure on the distal femur and thereby prevent or at least reduce the embolisation. The channel may be created from (and accessed via) a proximal end of the femur or a distal end of the femur. Furthermore, the methods disclosed herein may be selectively used with cemented or uncemented stems and with flat (blade-shaped) or rounded (conical) stems.

### Proximal Approach

According to a first aspect of the present disclosure, there is provided a method of preparing a femur for insertion of a prosthetic femoral stem component, the femur comprising a femoral head, a femoral neck, a trochanteric fossa, a greater trochanter and a femoral canal, the method comprising:
amputating the femoral head and a portion of the femoral neck;
excavating the trochanteric fossa;
preparing the femoral canal for insertion of the prosthetic femoral stem component such that a proximal end of the prosthetic femoral stem component is adjacent to a posterior cortical wall of the femoral canal, antero-medially to the excavated trochanteric fossa;
preparing the femoral canal for insertion of a vent tube in a vent path through an anterior portion of the femur, into the femoral canal and along an anterior cortical wall of the femoral canal to a position distal of a distal end of the prosthetic femoral stem component, when inserted in the femoral canal; and
inserting a vent tube into the vent path such that a tip of the vent tube is located in soft cancellous bone in the femoral canal, for use in venting the femoral canal during subsequent insertion of the prosthetic femoral stem component.

Embodiments of this aspect of the disclosure therefore provide a method for creating a channel (vent path) through the femur, for receipt of a vent tube from a proximal end of the femur. Once inserted, the vent tube can be attached to a surgical suction device to lower the pressure on the distal femur on insertion of a prosthetic femoral stem component, thereby preventing or at least reducing the risk of embolisation. The vent tube is inserted through an anterior portion of the femur and into the anterior femoral canal. The vent path may or may not pass through a hole in the greater trochanter. However, creating the vent path through the anterior of the greater trochanter is more convenient for a surgeon by keeping the tube out of the way during the majority of the procedure. This method may be particularly useful when flat (blade-shaped) stems are employed as there is unlikely to be enough material in the stem itself to safely accommodate a vent hole through the stem without risk of the stem fracturing and a flat stem allows space between the flat anterior femoral component and the cortical bone curvature.

The above method may be implemented for either cemented or uncemented stems. However, it is critical that the stem is inserted in the correct position in order to leave adequate space for the vent tube anteriorly of the stem. Note, the applicant has spent several years studying computed tomography (CT) scans of the anatomy of the femur in order to devise the proposed path for the vent tube, taking into the account the optimal position of the prosthetic femoral stem component. Accordingly, once inserted, the vent tube and the prosthetic femoral stem component may both be located, in a spaced-relationship, in a sagittal plane slicing in an anterior-posterior direction through the femur.

The method may comprise inserting the tip of the vent tube to a position distal of a tip of the prosthetic femoral stem component (which, for example, may comprise a flat or substantially flattened stem component), when inserted in the femur. In some cases, the vent tube may comprise side vent holes towards the tip of the vent tube and the side vent holes may also be located distal to the tip of the prosthetic femoral stem component.

The step of preparing the femoral canal for insertion of the vent tube may comprise creating a substantially straight vent path in the femur.

The step of preparing the femoral canal for insertion of the vent tube may comprise using one or more vented tools such as a vented reamer.

The step of preparing the femoral canal for insertion of the prosthetic femoral stem component may comprise using one or more vented tools such as a vented reamer, rasp or broach.

The step of preparing the femoral canal for insertion of the vent tube may be performed before, but preferably, after the step of preparing the femoral canal for insertion of the prosthetic femoral stem component.

The method may further comprise venting the femur, by operation of a suction device attached to the vent tube, whilst inserting the prosthetic femoral stem component.

The method may comprise directly or indirectly attaching a suction device to the vent tube. For example, the method may comprise attaching a first end of a flexible extension tube to the vent tube and attaching a second end of the flexible extension tube to the suction device. In some embodiments, the vent tube may have an entrance configured for attachment of a suction device and/or a flexible extension tube. For example, the entrance may be threaded internally and/or externally. The entrance may be enlarged compared to a remainder of the vent tube.

The step of amputating the femoral head and a portion of the femoral neck may comprise using any suitable surgical technique.

The step of excavating the trochanteric fossa may comprise using any suitable surgical technique (e.g. using an air-driven burr).

The vent tube may have a hollow passage there-through and may comprise one or more side holes in fluid communication with the hollow passage. The vent tube may comprise a plurality of said side holes. The one or more side holes may be provided in a region towards the tip of the vent tube (e.g. to be located below a tip of the prosthetic femoral stem component when inserted into the femur and/or below a cement restrictor, when in use). Additionally, one or more side holes may be provided in a proximal region of the vent tube (e.g. to be located within cancellous bone in the greater trochanter). The vent tube may be configured to have no side holes in a mid-section, which is configured to be disposed alongside the prosthetic femoral stem component in the femoral canal.

The vent tube may have a 6 mm outer diameter and a 4 mm diameter bore. The vent tube may have a 5 mm outer diameter and a 3 mm diameter bore. The vent tube may have a 4 mm outer diameter and a 2 mm diameter bore.

The vent tube may have a substantially circular cross-section. The vent tube may have a substantially oval cross-section. The vent tube may have a substantially D-shaped cross-section and may be orientated such that a curved portion of the vent tube is located along the anterior cortex wall of the femoral canal, and a flat portion of the vent tube is arranged to face the prosthetic femoral stem component (which may similarly have a flat facing surface) in the femoral canal. The vent tube may be formed of metal or polymethyl methacrylate (PMMA).

An alternative to using a vent tube with both distal and proximal side holes is to only use a vent tube with distal side holes but to additionally use a vent cannula inserted directly into the greater trochanter cancellous bone.

The method may comprise extracting the vent tube from the femur after the prosthetic femoral stem component has been inserted. For example, the method may comprise inserting a pole (e.g. a stiff wire or the like) into the vent tube and using a tool (such as pliers) to grip an exposed portion of the vent tube, with the pole therein, and withdrawing the vent tube and pole simultaneously from the vent path. Typically, this may only be done with a metal vent tube. Ideally, a metal vent tube would be used with an uncemented stem. Following removal of the metal vent tube, the proximal end of the vent canal is blocked with cancellous bone chips to prevent ingress of debris. Alternatively, the vent tube may be left in situ. For example, the vent tube may be formed of PMMA and used with a cemented stem implant.

When a cemented stem is used, the method may comprise inserting a cement restrictor into the femoral canal, distal of the prosthetic femoral stem component. The cement restrictor may be inserted with the vent tube pre-inserted to a desired depth in the cement restrictor (for example, with a part with vent side holes disposed below the cement restrictor and a part with no side holes disposed above the cement restrictor). When the vent tube is inserted to the desired depth in the cement restrictor, the vent tube and cement restrictor are inserted into the femoral canal together. Typically, the cement restrictor is constricted (i.e. tightened) in the femur when impacted and, consequently, the vent tube is effectively locked in position in the cement restrictor.

The cement restrictor may comprise a vent aperture for receipt of the vent tube there-through. The vent aperture may be provided towards an anterior edge of the cement restrictor. The cement restrictor may comprise a stem locator, to aid location of the tip of the prosthetic femoral stem component. The stem locator may comprise an indent configured for receipt of the tip of the prosthetic femoral stem component. The stem locator may be provided centrally on the cement restrictor. The cement restrictor may comprise one or more peripheral fins configured to flex on contact with the internal cortical wall for a snug fit therewith.

The method may comprise venting the femur, by operation of a suction device attached to the vent tube, before and whilst inserting liquid cement (typically using a cement gun), into a prepared cavity, above the cement restrictor, prior to insertion of the prosthetic femoral stem component into the cement-filled cavity.

When the cement around the prosthetic femoral stem has cured and the flow of fat and marrow is seen to stop through the clear plastic suction tubing, the PMMA vent tube is cut level with the top of the femur. Then the vent tube is plugged. The step of plugging the vent tube may comprise using a tool to insert a deformable plug to the level of the cement restrictor. The deformable plug may be formed of silicone. The deformable plug may comprise an internal screw-thread such that is can be screwed onto a tip of the tool, prior to insertion and then unscrewed from the tip of the tool once located in the vent aperture. The tool may be configured to compress the deformable plug in a transverse direction for insertion through the vent path and into the vent aperture, whereupon the tool may be configured to release the deformable plug, resulting in transverse expansion of the deformable plug for a secure fit within the vent aperture.

The method may comprise injecting liquid cement into the retained PMMA vent tube, above the deformable plug, to fill the vent tube between the cement restrictor and an entrance into the femur.

### Distal Approach

According to a second aspect of the present disclosure, there is provided a method of preparing a femur for hemi arthroplasty or total hip replacement, the femur comprising a lateral femoral condyle, a femoral canal and a proximal end, the method comprising: cutting into a non-distal portion of the lateral femoral condyle to form a part of a vent path;
introducing a cutting tool into the part of the vent path, the cutting tool comprising a hollow tube having a cutting blade mounted at a tip thereof;
inserting a rod into the hollow tube of the cutting tool, the rod having a substantially straight shaft and a curved tip configured to guide the cutting blade in a curved path;
manoeuvring the cutting tool, in conjunction with the inserted rod, in order to extend the vent path, along the curved path defined by the curved tip, until the cutting blade is substantially aligned with a longitudinal axis of the femoral canal;
removing the rod and advancing the cutting tool along the femoral canal, to extend the vent path in a direction towards the proximal end of the femur;
removing the cutting tool from the vent path; and
inserting a vent tube into the vent path such that a tip of the vent tube is located in soft cancellous bone in the femoral canal, for use in venting the femur during subsequent hemi arthroplasty or total hip replacement.

Embodiments of this aspect of the disclosure therefore provide a method for creating a vent path through the femur, for receipt of a vent tube from a distal end of the femur. Once inserted, the vent tube can be attached to a surgical suction device to lower the pressure on the distal femur, either during hemi arthroplasty or total hip replacement (e.g. on insertion of a prosthetic femoral stem component), thereby preventing or at least reducing the risk of embolisation. This method may be useful when either flat (blade-shaped) stems or rounded (conical) stems are employed in total hip replacement because the vent path in this case may be entirely disposed distally of the prosthetic femoral stem component.

The above method may be implemented for either cemented or uncemented stems.

The method may comprise inserting the tip of the vent tube to a position distal of a tip of the prosthetic femoral stem component, when inserted in the femur.

The method may comprise extending the vent path into a middle region of the femur (e.g. a middle third of the femur).

The method may further comprise inserting a guide wire into the vent path prior to inserting the vent tube into the vent path. The guide wire may be inserted such that it extends beyond an end of the vent path. The vent tube may be threaded over the guide wire and pushed along the guide wire to extend the vent path. Once the vent tube is in position, the guide wire may be removed. The method may comprise imaging the femur to monitor the position of the cutting tool and/or guide wire in order to ensure accurate creation of the vent path. The imaging may comprise digital fluoroscopy or computerized navigation.

The method may further comprise venting the femur, by operation of a suction device attached to the vent tube, whilst preparing the femur for insertion of the prosthetic femoral stem component.

The method may further comprise venting the femur, by operation of a suction device attached to the vent tub, whilst inserting the prosthetic femoral stem component.

The method may comprise directly or indirectly attaching a suction device to the vent tube. For example, the method may comprise attaching a first end of a flexible extension tube to the vent tube and attaching a second end of the flexible extension tube to the suction device. In some embodiments, the vent tube may have an entrance configured for attachment of a suction device and/or a flexible extension tube. For example, the entrance may be threaded internally and/or externally. The entrance may be enlarged compared to a remainder of the vent tube.

The method may further comprise inserting a vent cannula into cancellous bone in the greater trochanter, to aid with venting a proximal region of the femur during subsequent hemi arthroplasty or total hip replacement.

In some embodiments, the second aspect of the disclosure may be performed in a separate procedure, prior to a hemi arthroplasty or total hip replacement procedure.

The rod may be formed of metal and may be configured to substantially hold its shape when in use.

The hollow tube of the cutting tool may be formed of a resilient material configured to substantially conform to the shape of the rod, when inserted therein, but to otherwise revert to a rest position which is substantially in the form of a circular cylinder.

The cutting tool may be provided with a handle extending at an angle to the longitudinal axis of the hollow tube, when in the rest position. The angle may be such that the handle is substantially aligned with the longitudinal axis of the femoral canal, when the cutting blade is substantially aligned with a longitudinal axis of the femoral canal, after the cutting blade has followed the curved path defined by the curved tip of the rod. In other words, the angle of the handle with respect to the hollow tube may be related to (e.g. supplementary to) an angle of the curved tip of the rod with respect to the shaft of the rod.

The handle may be provided with a blade for penetrating the lateral femoral condyle.

Rotation of the handle (e.g. about the longitudinal axis of hollow tube) may advance the cutting blade along the femoral canal.

The vent tube has a hollow passage there-through and may comprise one or more side holes in fluid communication with the hollow passage. The vent tube may comprise a plurality of said side holes. The one or more side holes may be provided in a region towards the tip of the vent tube. The vent tube may have a 6 mm outer diameter and a 4 mm diameter bore. The vent tube may have a 5 mm outer diameter and a 3 mm diameter bore. The vent tube may have a 4 mm outer diameter and a 2 mm diameter bore.

### Cemented Stems

According to a third aspect of the present disclosure, there is provided a prosthetic femoral stem component comprising:
a substantially longitudinal stem portion;
a neck portion having a longitudinal axis intersecting the longitudinal stem portion; and
a vent hole extending through the longitudinal stem portion from a proximal surface to a distal tip;
wherein the vent hole comprises an enlarged portion adjacent to the proximal surface, for accommodating an end of a suction device during use.

Thus, embodiments of this disclosure provide a prosthetic femoral stem component comprising a vent hole there-through, which includes an enlarged portion for accommodating an end of a suction device during use.

It should be noted that space to accommodate a vent hole is limited in a prosthetic femoral stem component, particularly at or in the region of, the distal tip of the stem. As such, a small diameter hole is preferred, however, it may be challenging to effectively attach a suction device to such a small hole and therefore it is advantageous for the hole to have an enlarged portion at a proximal end, at which location the stem is generally more substantial.

The enlarged portion may be configured to directly or indirectly accommodate an end of a suction device during use. For example, in some embodiments, a flexible tube connector may be inserted into the enlarged end and the suction device attached to an opposite end of the flexible tube connector.

The stem portion may have a substantially round transverse section and the vent hole may pass through a center of the transverse section of the stem portion in a region approximately one third of a distance from the distal tip to the proximal surface. This arrangement ensures maximum material surrounds the vent hole in a region typically vulnerable to fracture.

The enlarged portion may comprise a threaded bore. The threaded bore may useful for attaching other components. For example, the threaded bore may be used to attach a flexible tube forming an extension to a suction device in situations where there is restricted access for the suction device to be directly connected to the enlarged portion.

The prosthetic femoral stem may further comprise a stopper configured for secure insertion into the enlarged portion. The stopper may be secured in the enlarged portion to seal the vent hole after the femoral stem component has been securely fixed in the femur.

The stopper may comprise an external thread for mating with the threaded bore of the enlarged portion.

The stopper may be in the form of a hex socket screw-in plug.

The prosthetic femoral stem may further comprise a tapered portion connecting the enlarged portion to a remainder of the vent hole.

The vent hole may have a diameter of at least 3 mm. In some cases, the vent hole may have a diameter of approximately 4 mm. The enlarged portion may have a diameter of approximately 7 mm, in order to accommodate an end of a traditional disposable plastic surgical suction device having an external diameter of approximately 7 mm.

The vent hole may be substantially straight.

According to a fourth aspect of this disclosure, there is provided a method of cementing a prosthetic femoral stem component, as described above in relation to the third aspect, into a femur, the method comprising: inserting a cement restrictor, having a bore there-through, into a femoral canal at a position determined to be below a position of the distal tip of the prosthetic femoral stem component, when located therein;
inserting an extension tube into the bore of the cement restrictor;
operating a suction device through a proximal end of the extension tube whilst injecting liquid cement into the femoral canal around the extension tube;
removing the suction device from the proximal end of the extension tube;
inserting the distal tip of the prosthetic femoral stem component into the proximal end of the extension tube; the connection being a snap fit to prevent disconnection as the stem is inserted into the liquid cement. inserting the suction device into the enlarged portion of the vent hole of the prosthetic femoral stem component; and
operating the suction device whilst inserting the prosthetic femoral stem component into the femoral canal and waiting for the cement to cure to fix the prosthetic femoral stem component in the femoral canal.

The extension tube may comprise an enlarged portion at the proximal end, for accommodating an end of the suction device and, separately, for a snap-fit insertion of the distal tip of the of the prosthetic femoral stem component.

The enlarged portion may comprise a threaded bore. The threaded bore may be useful for attaching other components. For example, the threaded bore may be used to attach a flexible tube forming an extension to a suction device in situations where there is restricted access for the suction device to be directly connected to the enlarged portion.

The extension tube may have an internal diameter of at least 3 mm. In some cases, the extension tube may have an internal diameter of approximately 4 mm.

The extension tube may be substantially straight, but flexible and manufactured from PMMA.

The cement restrictor may comprise a proximal plug portion and a perforated tube extending distally therefrom, wherein the bore extends through the plug portion and the perforated tube. The perforated tube may be configured to accommodate the extension tube, when the prosthetic femoral stem component is fixed in the femoral canal. The perforated tube may be provided in differing lengths to suit different patients.

### Cemented Stems for Distal Migration

According to a fifth aspect of this disclosure, there is provided a prosthetic femoral stem comprising: a neck portion and a stem portion; the stem portion having a distal tip and a vent hole through the stem portion from a proximal surface to the distal tip; the stem portion comprising a concavely curved medial surface having a single radius of curvature extending from a junction between the neck portion and the stem portion to a region a third of the distance from the distal tip; and wherein the vent hole is located centrally through the stem portion in said region.

Any features described above in relation to the third aspect of the present disclosure may also apply to the fifth aspect of the disclosure.

### Uncemented Stems

According to a sixth aspect of this disclosure, there is provided a method of implanting an uncemented prosthetic femoral stem component, as described above in relation to the third aspect, into a femur, the method comprising:
preparing the femur for insertion of a round section prosthetic femoral stem component; inserting a suction device into the enlarged portion of the vent hole of the prosthetic femoral stem component; and operating the suction device whilst inserting the prosthetic femoral stem component into the femur.

### Instruments

According to a sixth aspect of the present disclosure, there is provided a set of instruments for use in preparing a femur for hemi arthroplasty or total hip replacement, comprising one or more of:
a vent tube as described above;
a cutting tool and rod as described above;
a cement restrictor as described above; and
a tool for inserting a deformable plug, and said deformable plug, as described above.

One or more of the instruments may be provided in a disposable pack for single use.

### BRIEF DESCRIPTION OF THE PREFERRED EMBODIMENTS

Some embodiments of the disclosure will now be described by way of example only and with reference to the accompanying drawings, in which:

FIG. 1 shows a graph illustrating the survival probability of patients fitted with cemented total hip replacement (THR) stems, uncemented total hip replacement (THR), and hip resurfacing.

FIG. 2A shows an echocardiogram of a heart before any surgery to the femur.

FIG. 2B shows an echocardiogram following traditional insertion of a cemented stem hip arthroplasty.

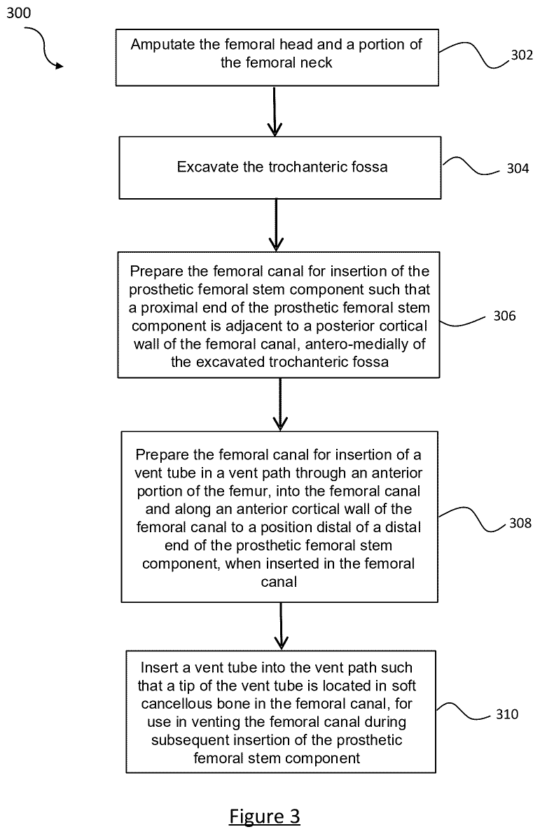

FIG. 3 shows a flow diagram illustrating a method of preparing a femur for insertion of a prosthetic femoral stem component;

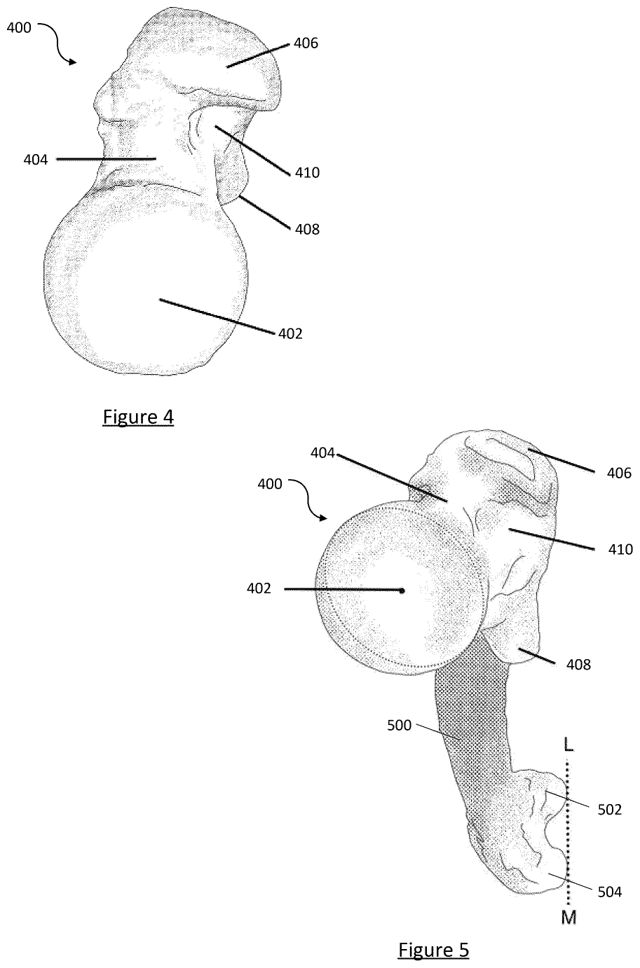

FIG. 4 shows a view of the top of a femur from above;

FIG. 5 shows a view of the femur from a posterior aspect;

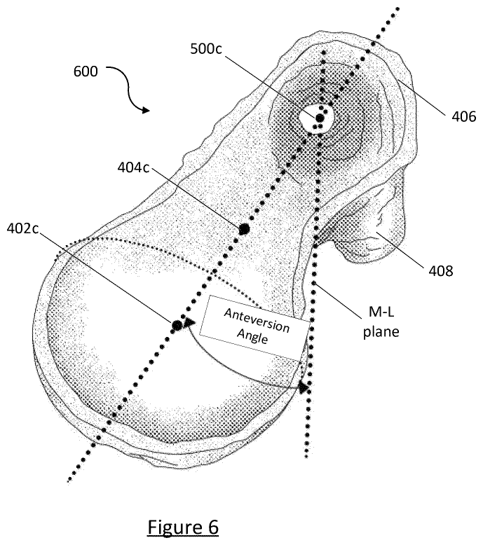

FIG. 6 shows a transverse section taken centrally through the femoral head and femoral neck;

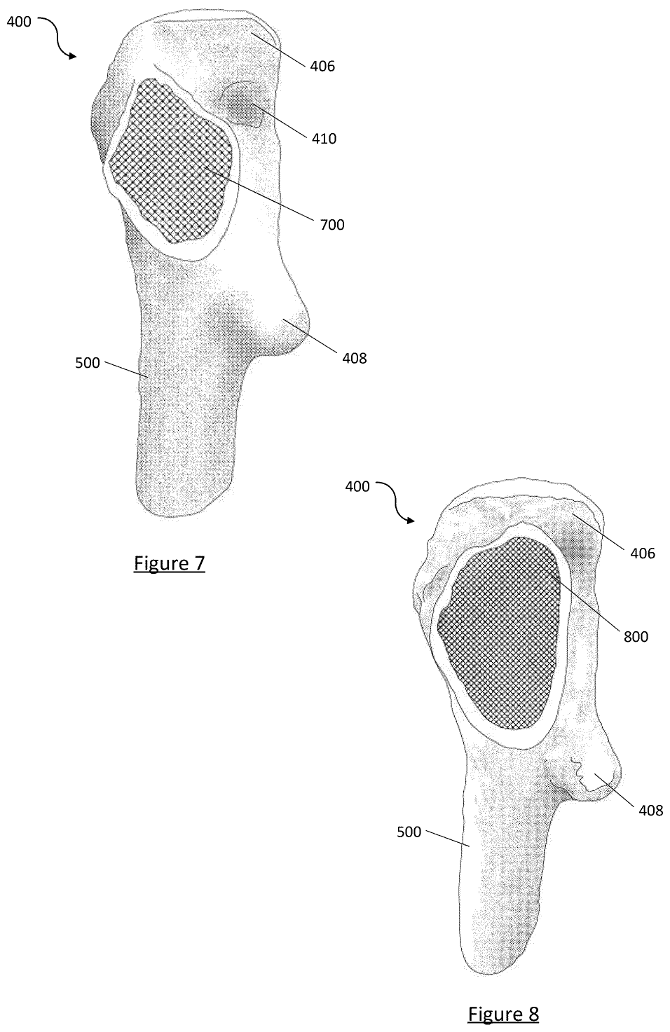

FIG. 7 shows a side view of the top of a femur after the femoral head and femoral neck have been resected;

FIG. 8 shows a view similar to that of FIG. 7 but with the trochanteric fossa excavated;

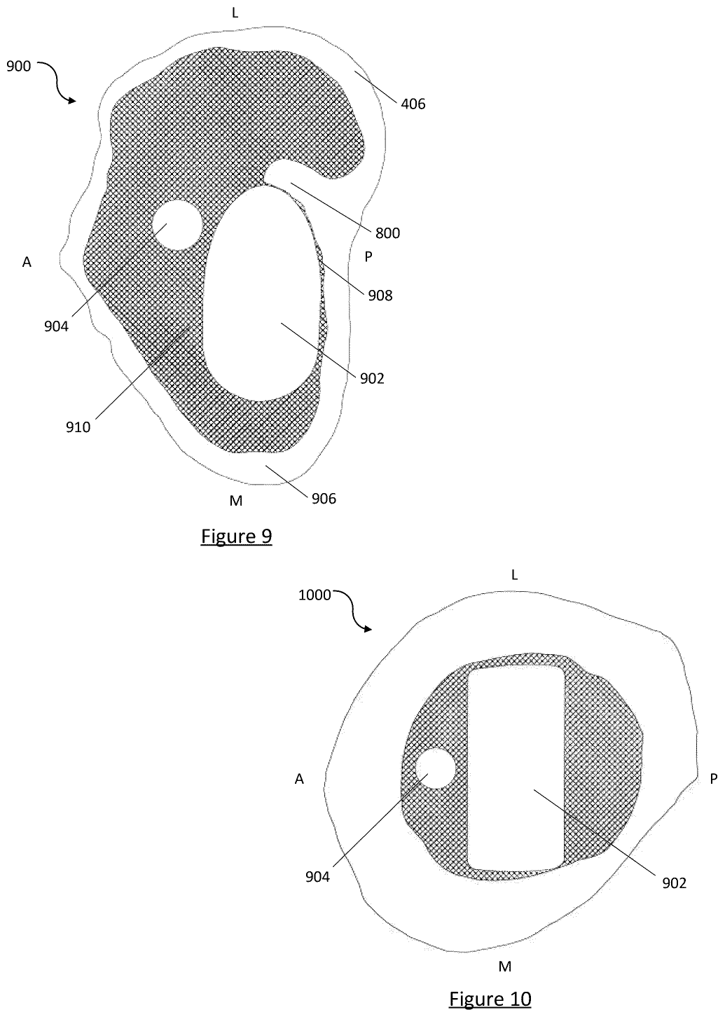

FIG. 9 shows a transverse section taken through the excavated trochanteric fossa and with a substantially flat stem prosthetic femoral stem component and vent tube in situ;

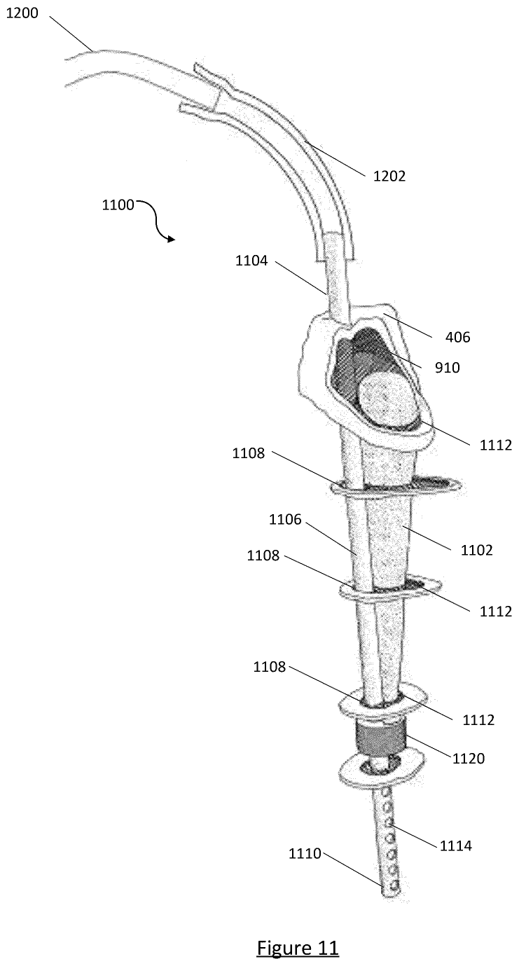

FIG. 10 shows a transverse section taken through the femur at a position half-way down a different design of prosthetic femoral stem component having a flat stem.

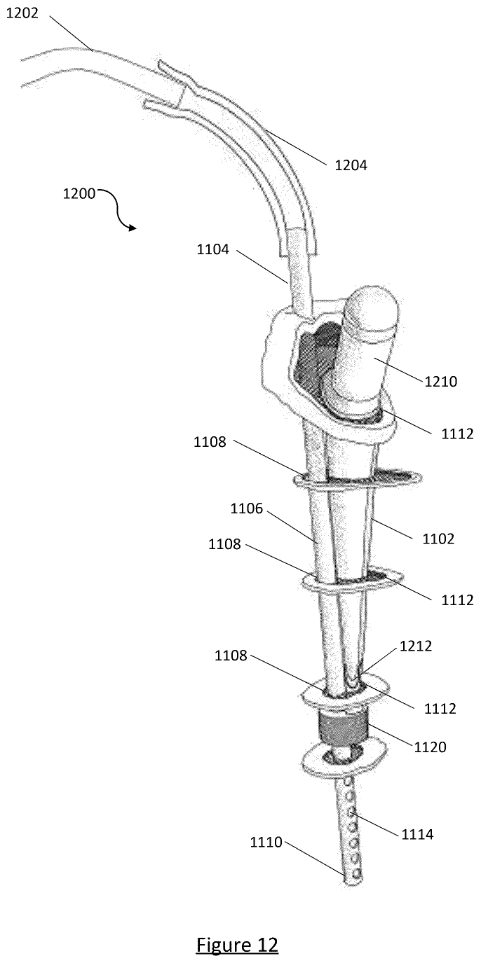

FIG. 11 shows a set up for venting a distal femoral canal during a procedure for inserting cement for a cemented prosthetic femoral stem component. Here a PMMA vent tube is being used.

FIG. 12 shows a set up for venting a distal femoral canal during a procedure for inserting a cemented prosthetic femoral stem component into the cement of FIG. 11;

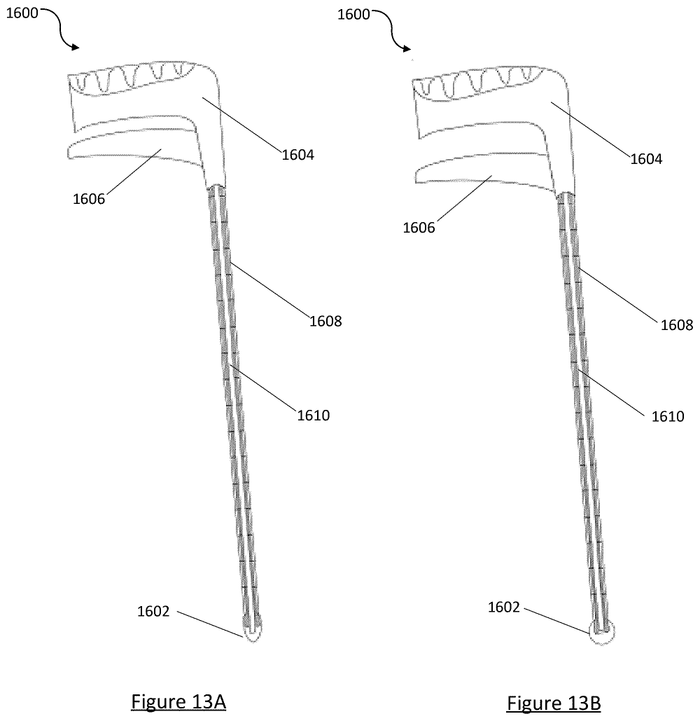

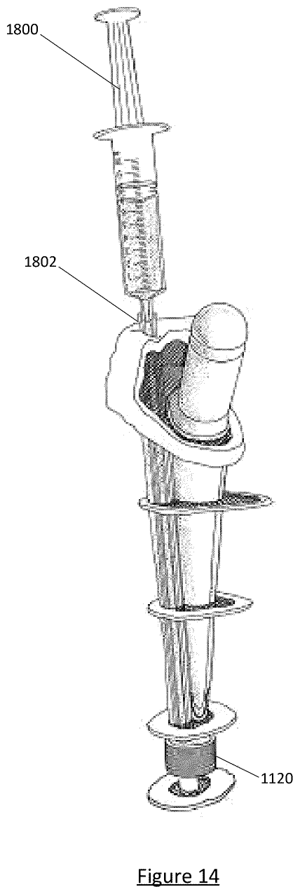

FIG. 13A shows a tool for inserting a deformable plug into a vent aperture in a cement restrictor, with the deformable plug in a compressed configuration for insertion;

FIG. 13B shows the tool of FIG. 13A, with the deformable plug in an expanded configuration for blockage;

FIG. 14 shows injection of low viscosity cement into the plugged PMMA vent tube.

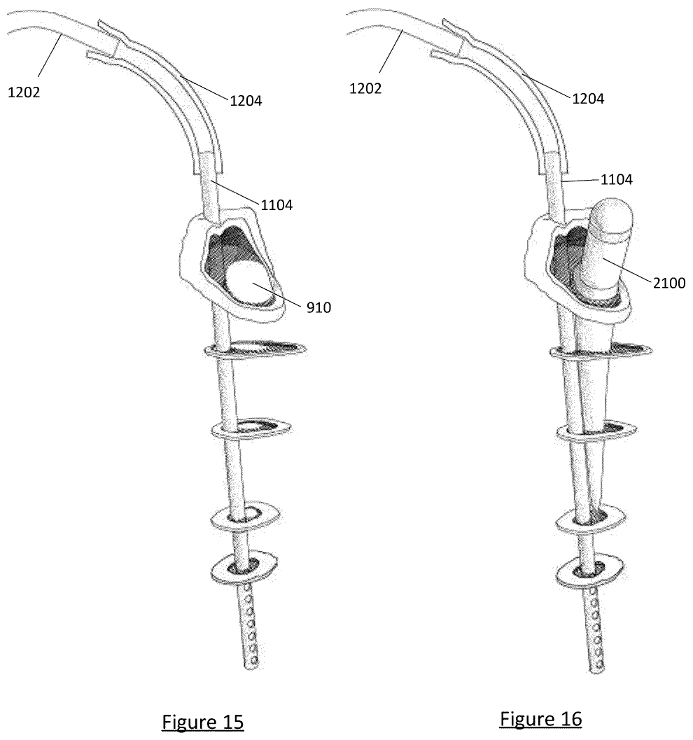

FIG. 15 shows a metal vent tube inserted into a proximal end of a femur and the femoral canal prepared for insertion of an uncemented prosthetic femoral stem component;

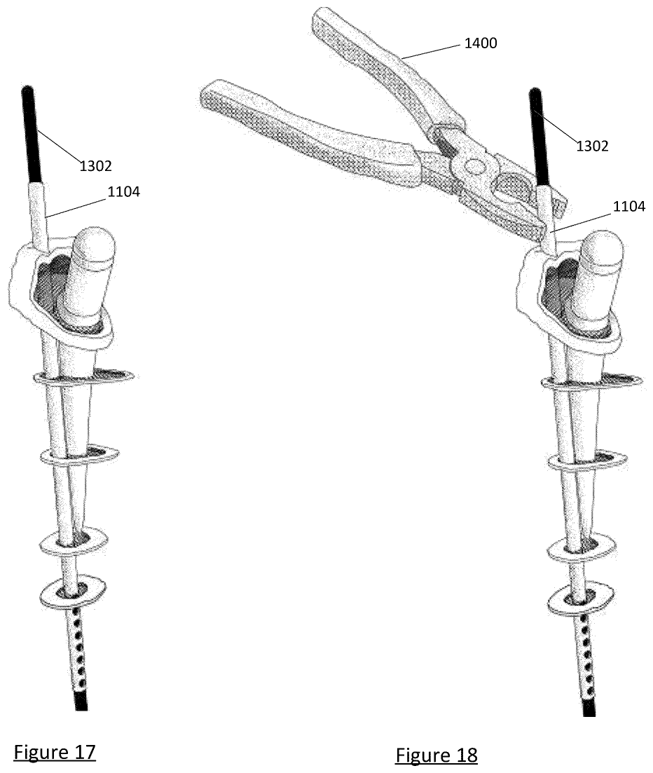

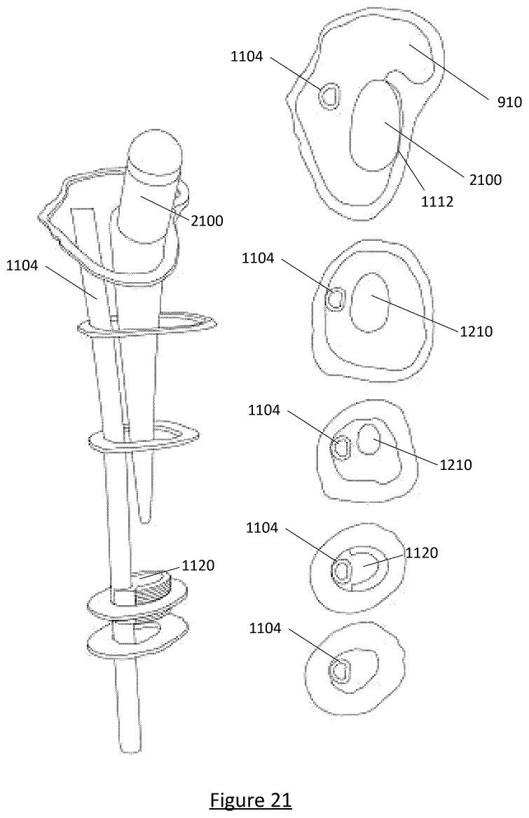

FIG. 16 shows a suction device attached to the metal vent tube of FIG. 20 during insertion of the uncemented prosthetic femoral stem component;

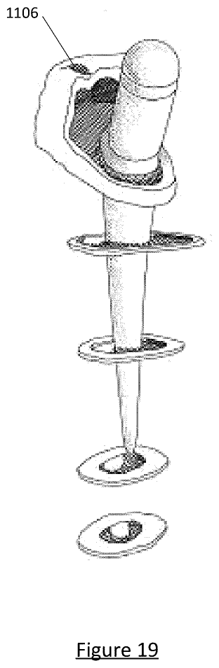

FIG. 17 shows insertion of a pole into the vent tube of FIG. 16 for extraction of the vent tube;

FIG. 18 shows removal of the pole and metal vent of FIG. 16 tube using pliers;

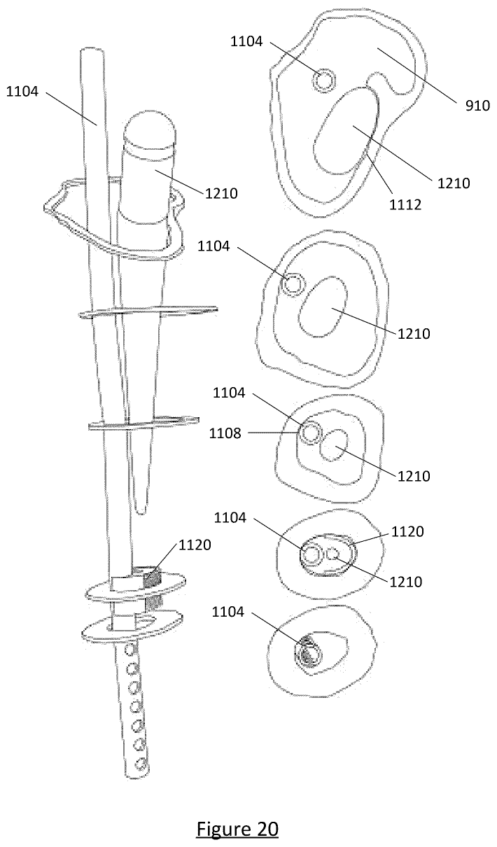

FIG. 19 shows the open vent path after removal of the metal vent tube. The proximal end of the vent path is blocked with cancellous bone chips.

FIG. 20 shows a circular vent tube in position during a cemented stem total hip arthroplasty;

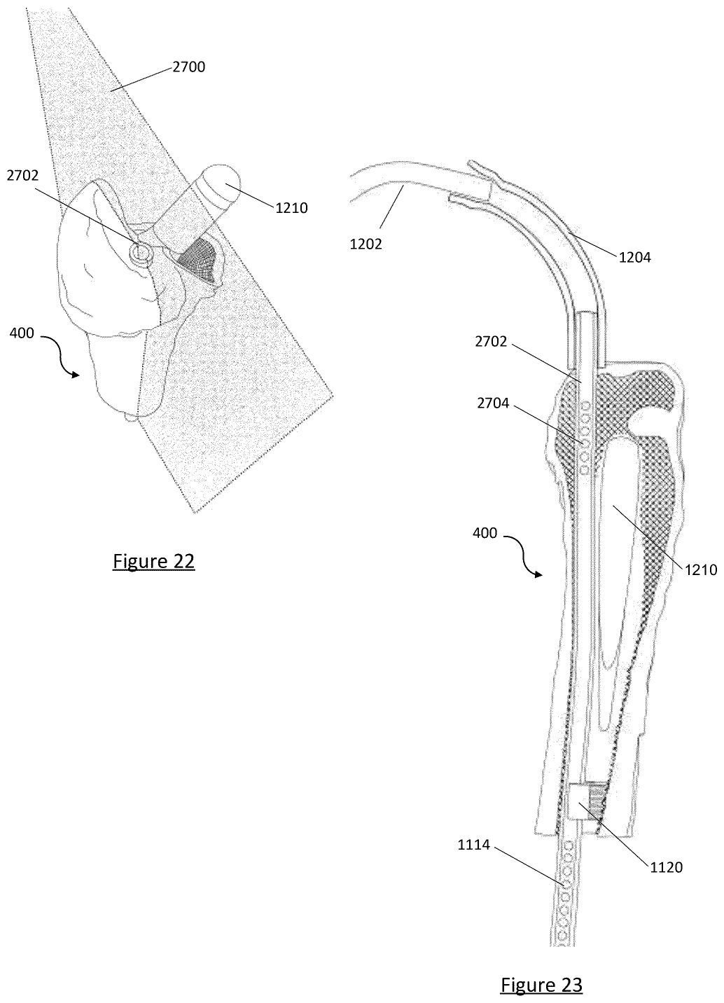

FIG. 21 shows a D-shaped vent tube in position after insertion of a cemented prosthetic femoral stem component;

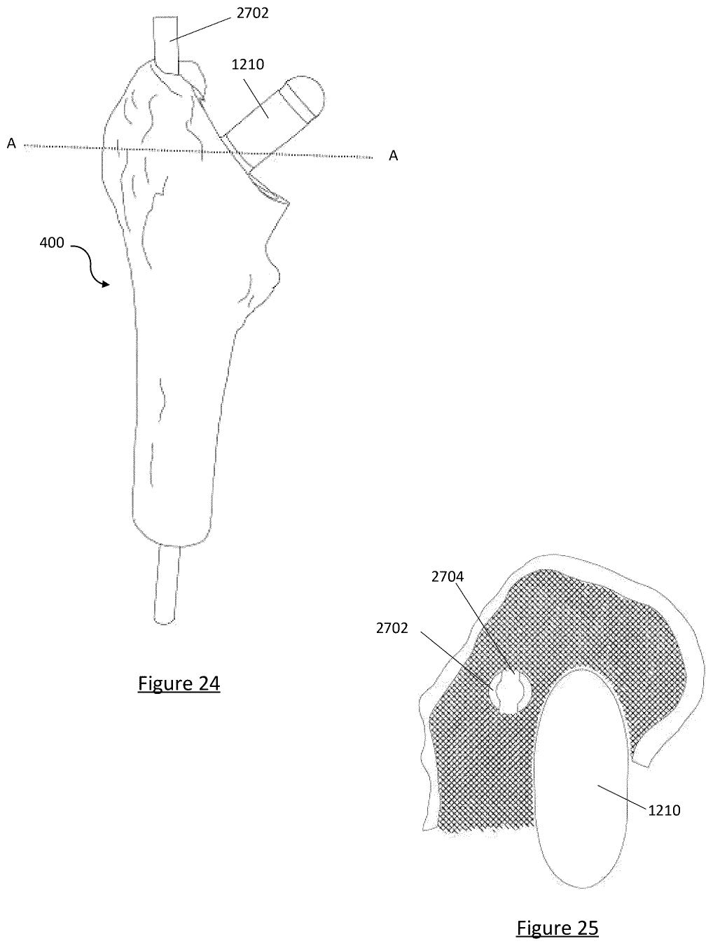

FIG. 22 illustrates a vertical plane through a femur to illustrate another vent tube design;

FIG. 23 shows a vertical section corresponding to the plane of FIG. 22 and with an extension tube and suction device attached to the vent tube;

FIG. 24 illustrates a transverse plane AA through the femur of FIGS. 22 and 23;

FIG. 25 shows a transverse section corresponding to the plane AA of FIG. 24, illustrating side holes in a proximal portion of the vent tube;

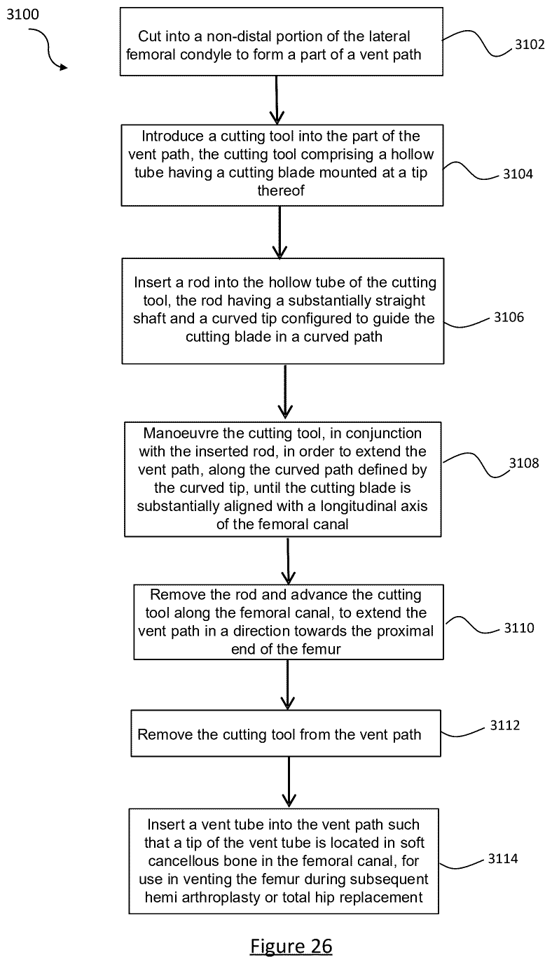

FIG. 26 shows a flow diagram illustrating a method of preparing a femur for hemi arthroplasty or total hip replacement;

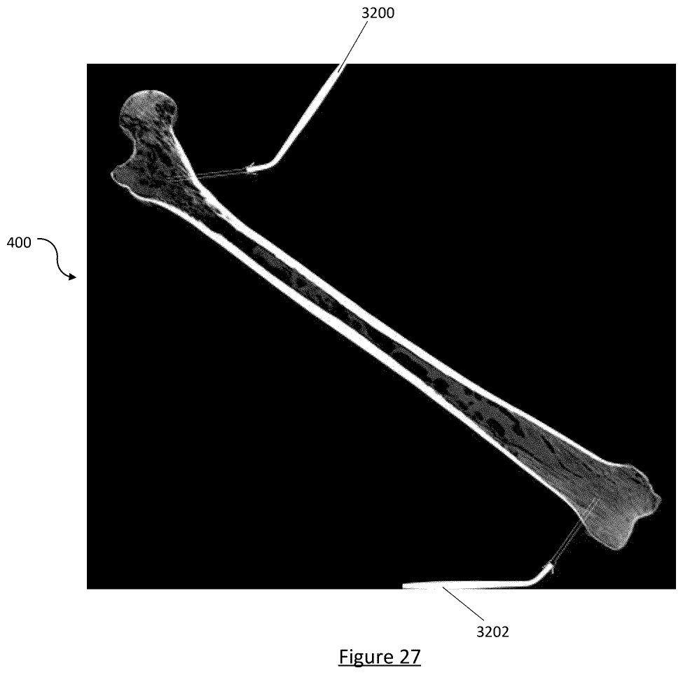

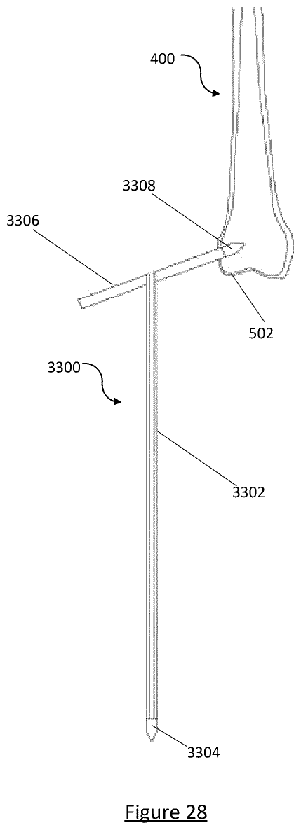

FIG. 27 shows a CT scan of a femur with vent cannulas inserted in both the proximal and distal ends of the femur;

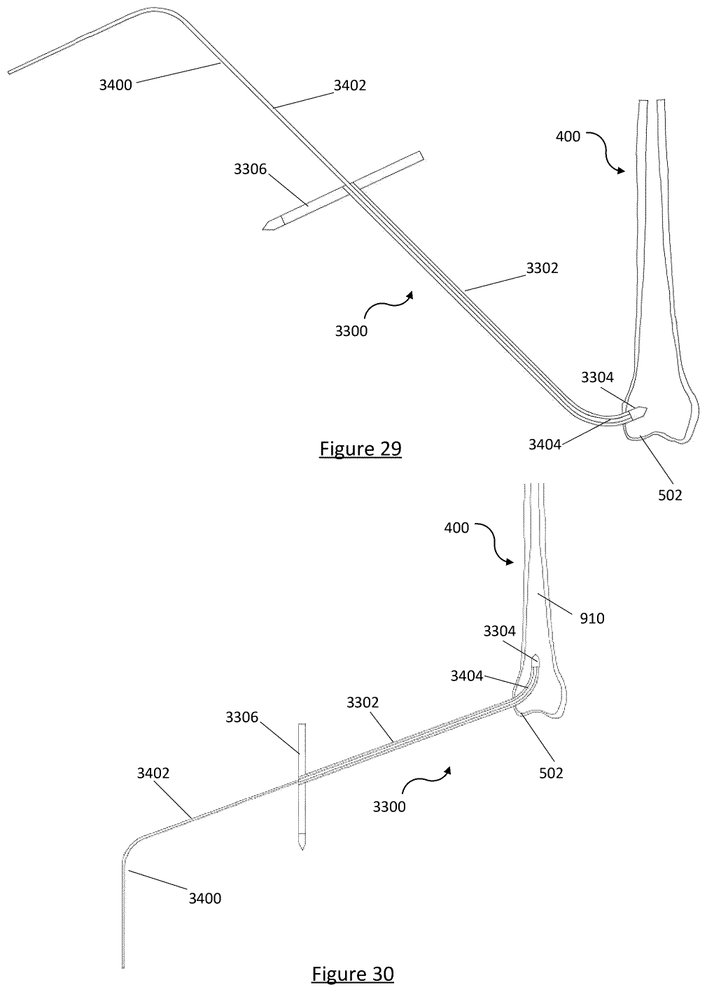

FIG. 28 shows an initial cut being made into the lateral femoral condyle;

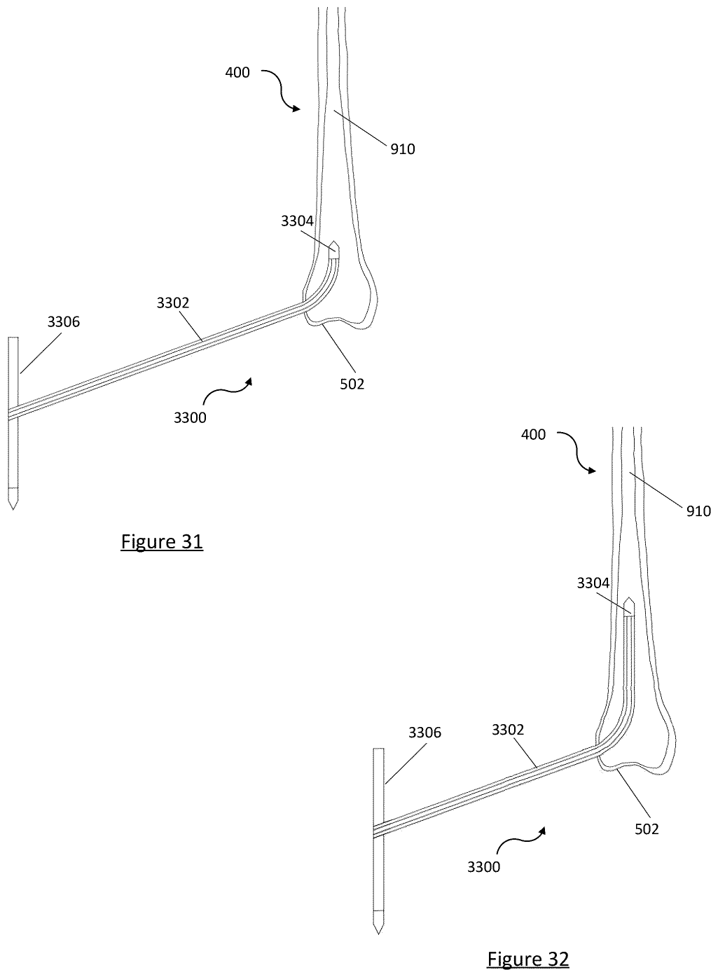

FIG. 29 shows a cutting tool and rod being inserted into the initial cut;

FIG. 30 shows the cutting tool and rod after the initial cut has been extended in a curved path;

FIG. 31 shows a view similar to that of FIG. 30 but with the rod removed;

FIG. 32 shows the cutting tool advanced further up the femoral canal;

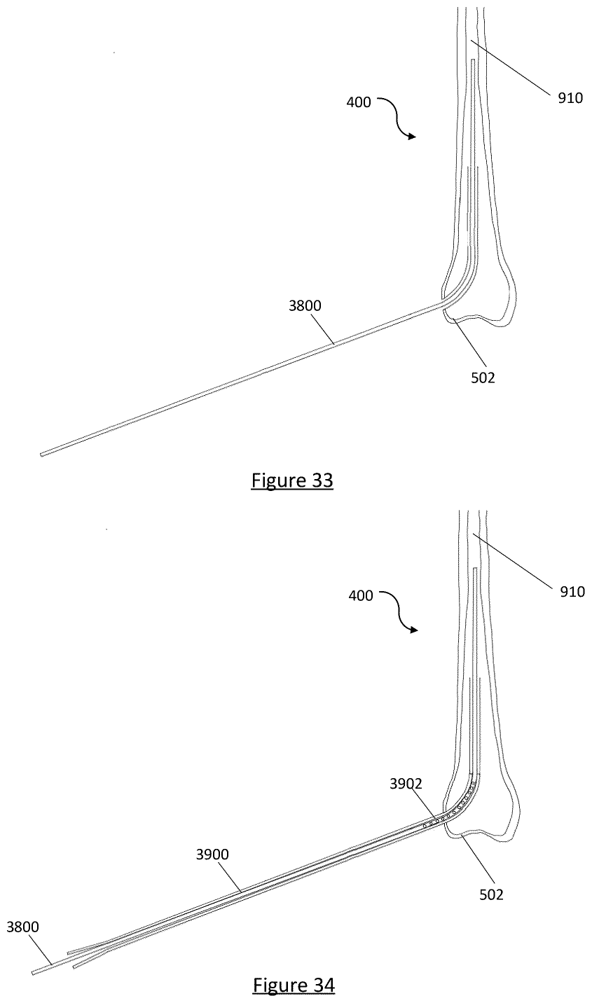

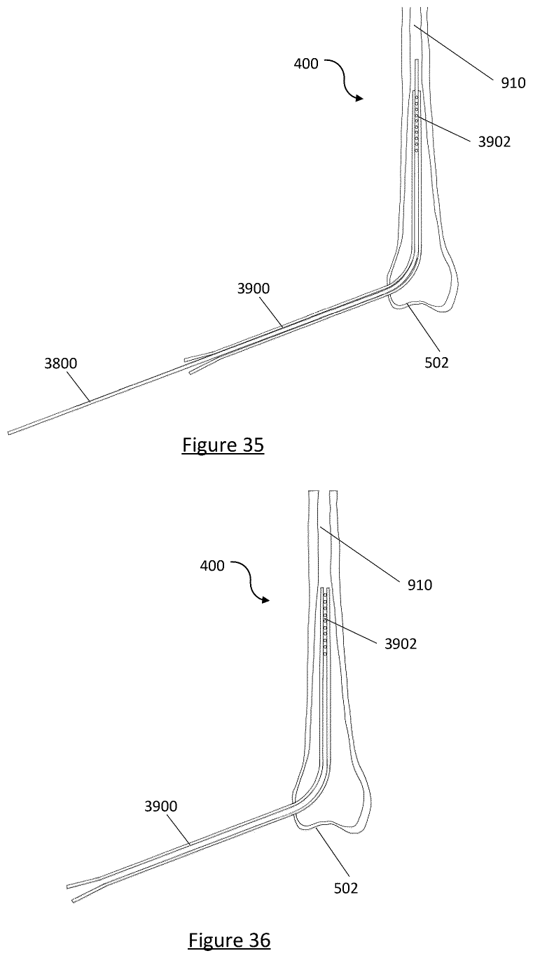

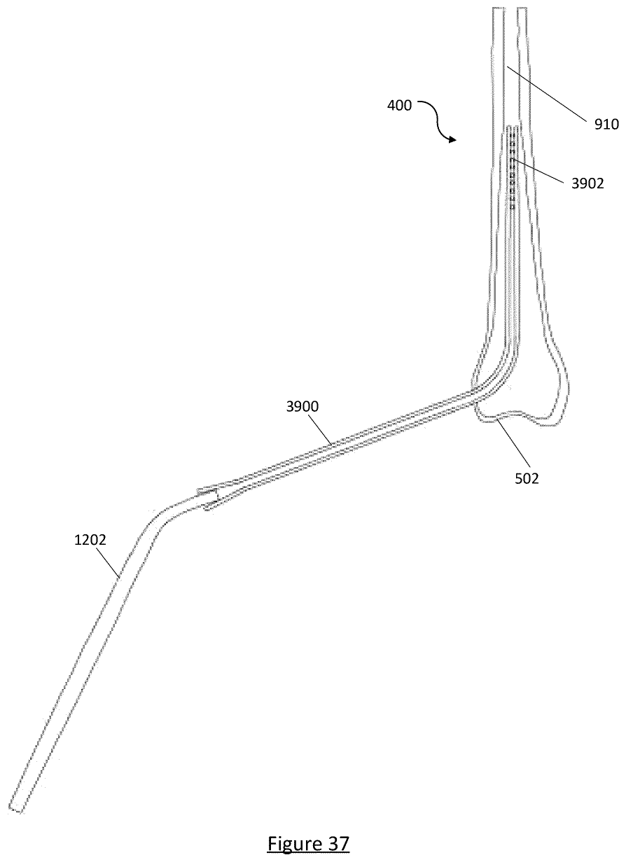

FIG. 33 shows a guide wire inserted along and beyond the created vent path;

FIG. 34 shows a vent tube being threaded along the guide wire;

FIG. 35 shows the vent tube advanced along the guide wire;

FIG. 36 shows the vent tube in position after removal of the guide wire; and

FIG. 37 shows a suction device attached to the vent tube for venting the femur.

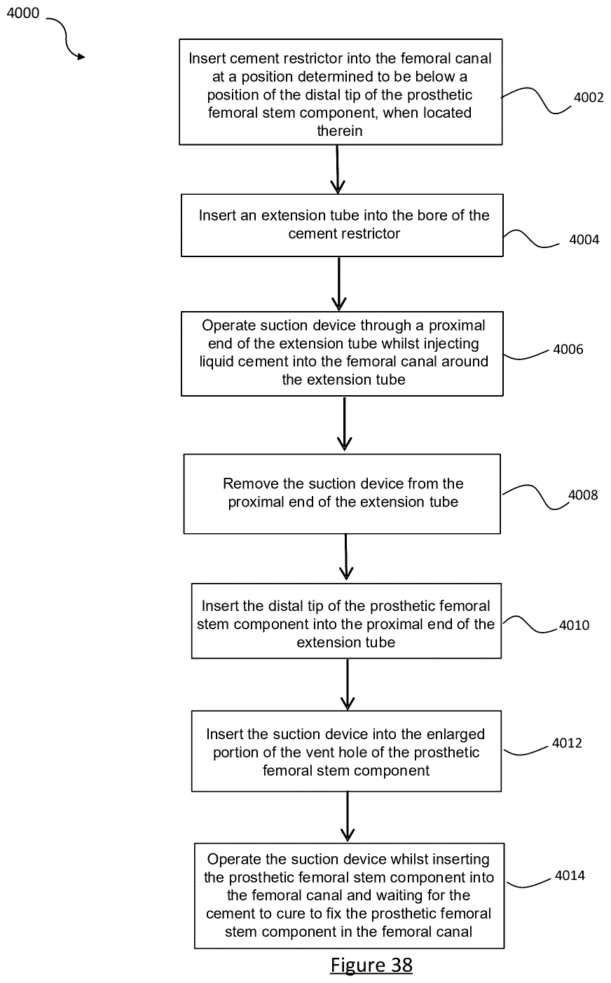

FIG. 38 shows a flow diagram illustrating a method of cementing a prosthetic femoral stem component into a femur, in accordance with the present disclosure.

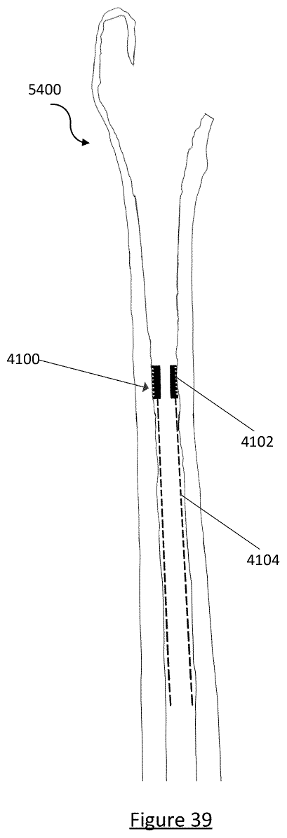

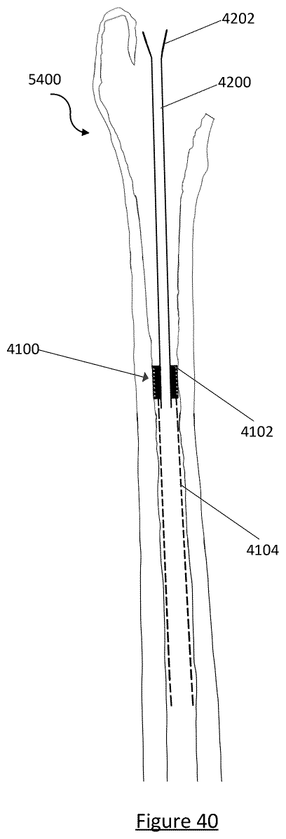

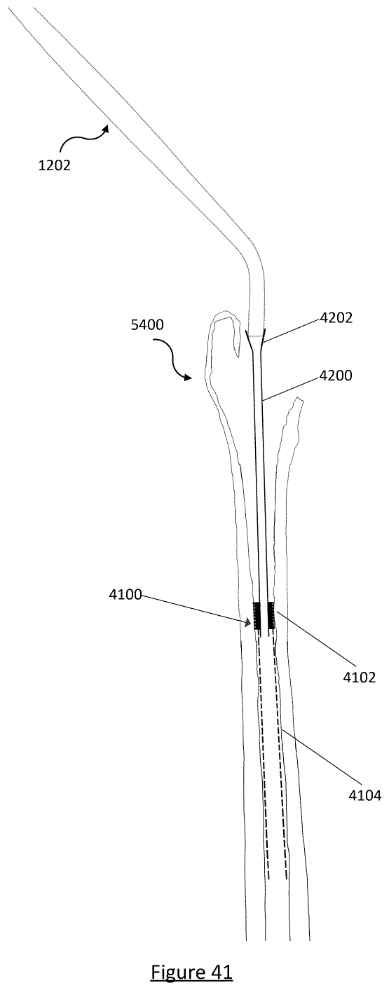

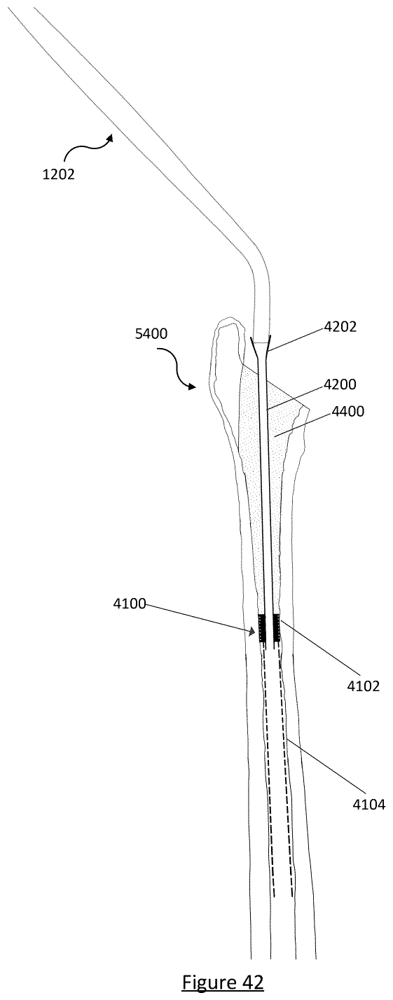

FIG. 39 shows a longitudinal cross-sectional view of a resected femur with a cement restrictor inserted therein, in accordance with the present disclosure.

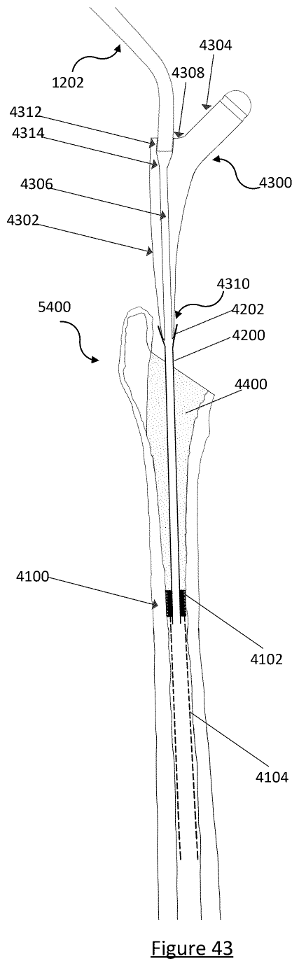

FIG. 40 shows a view similar to that of FIG. 39 but with an extension tube inserted into the cement restrictor, in accordance with the present disclosure.

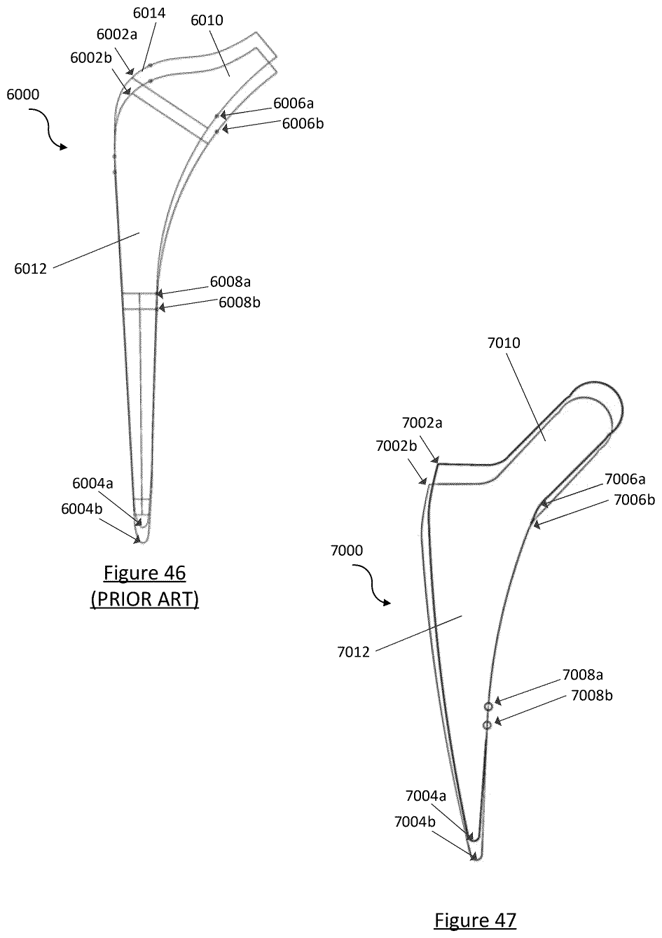

FIG. 41 shows a view similar to that of FIG. 44 but with a suction device inserted in the extension tube, in accordance with the present disclosure.

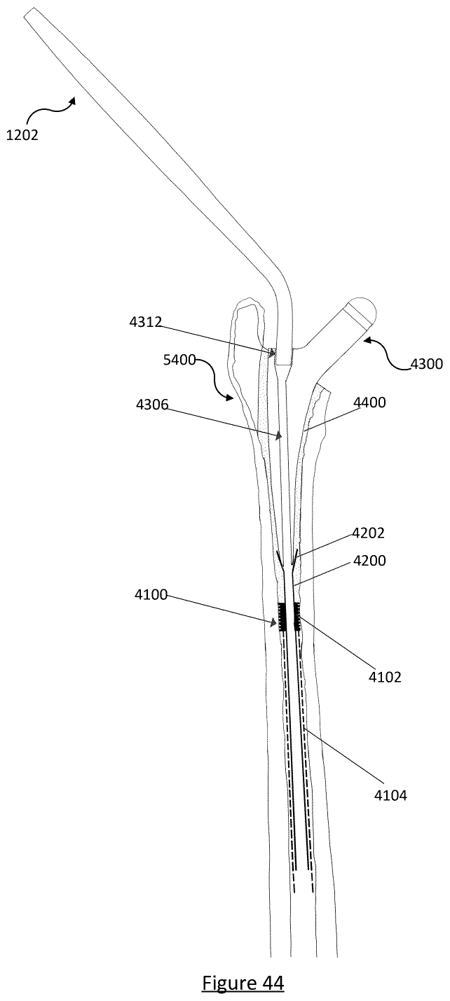

FIG. 42 shows a view similar to that of FIG. 41 but with liquid cement provided in the femoral canal, around the extension tube, in accordance with the present disclosure.

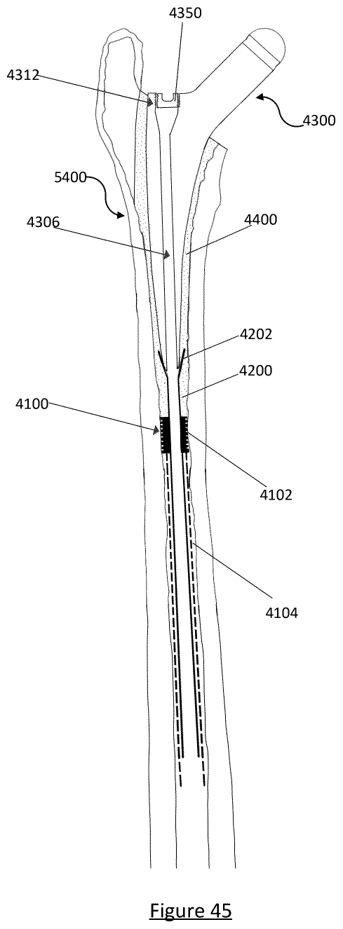

FIG. 43 shows a view similar to that of FIG. 42 but with a tip of a prosthetic femoral stem component inserted into the extension tube and the suction device attached to the prosthetic femoral stem component, in accordance with the present disclosure.

FIG. 44 shows a view similar to that of FIG. 43 but with the prosthetic femoral stem component inserted into the femoral canal, in accordance with the present disclosure.

FIG. 45 shows a view similar to that of FIG. 44 but with the suction device removed and a threaded stopper inserted into the vent hole of the prosthetic femoral stem component, in accordance with the present disclosure.

FIG. 46 shows forced distal migration of a known prosthetic femoral stem component, during use.

FIG. 47 shows forced distal migration of a prosthetic femoral stem component, according to the disclosure, during use, although for clarity a vent hole is not illustrated.

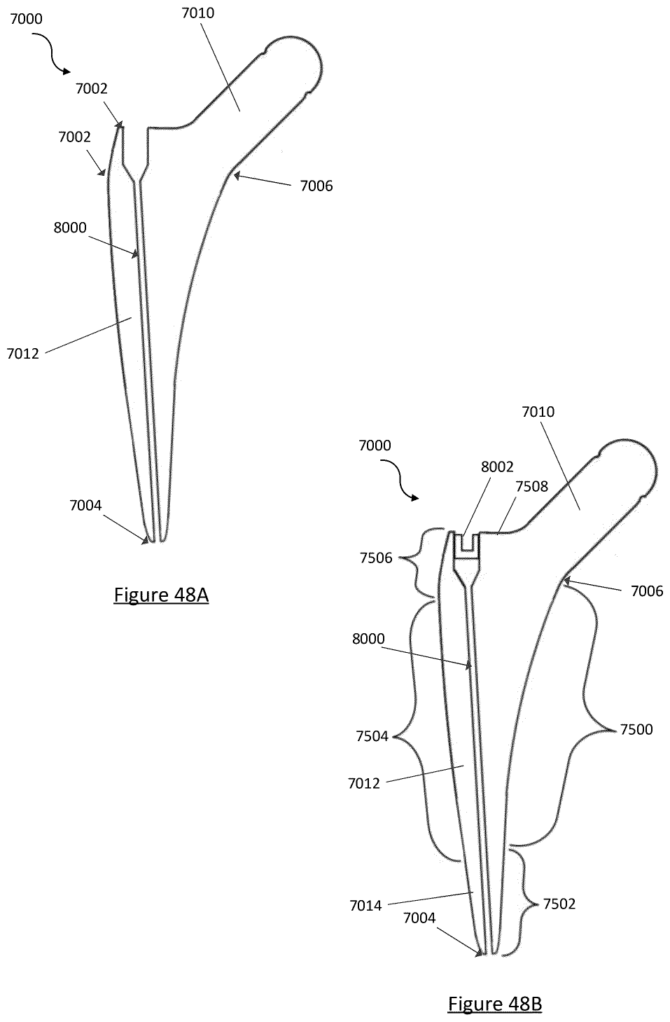

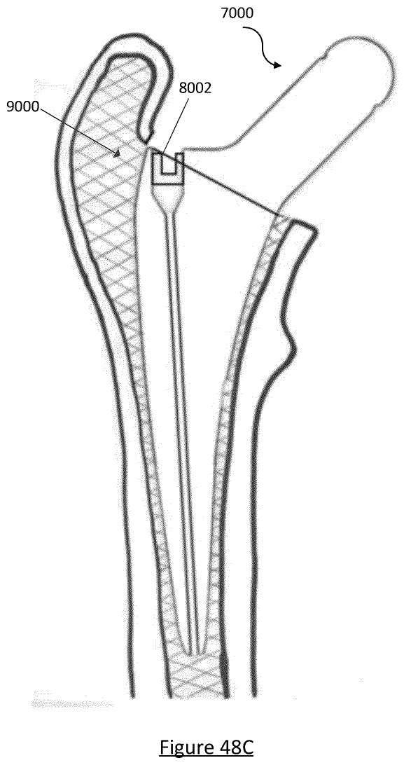

FIGS. 48A, 48B and 48C show, respectively, the prosthetic femoral stem component of FIG. 47, including a vent hole, with a plugged vent hole and when implanted in a resected femur.

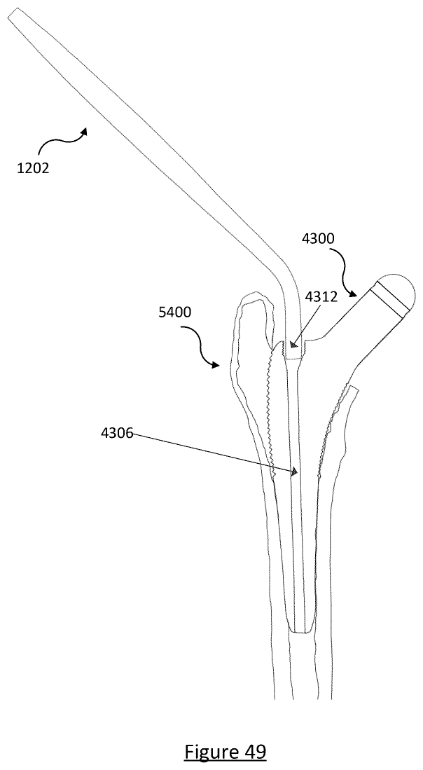

FIG. 49 shows a longitudinal cross-sectional view of a resected femur with an uncemented round section, canal filling, vented prosthetic femoral stem component inserted therein and with a suction device attached to the prosthetic femoral stem component, in accordance with the present disclosure.

### DETAILED DESCRIPTION OF THE PREFERRED EMBODIMENTS

Generally speaking, the disclosure provides methods for improved patient outcomes following hip arthroplasty. The over-riding aim is to provide methods for venting at least the distal femoral canal during stem implantation in order to lower the pressure on the distal femur and thereby prevent or at least reduce the embolisation. The principles of the disclosure may be applied to cemented stems, uncemented stems and surgical instruments used for preparing a femur for insertion of a prosthetic femoral stem component.

### Proximal Approach

FIG. 3 shows a flow diagram illustrating a method 300 of preparing a femur for insertion of a prosthetic femoral stem component, the femur comprising a femoral head, a femoral neck, a trochanteric fossa, a greater trochanter and a femoral canal.

The method 300 comprises a step 302 of amputating the femoral head and a portion of the femoral neck and a step 304 of excavating the trochanteric fossa. The method further comprises a step 306 of preparing the femoral canal for insertion of the prosthetic femoral stem component such that a proximal end of the prosthetic femoral stem component is adjacent to a posterior cortical wall of the femoral canal, antero-medially of the excavated trochanteric fossa. The method further comprises a step 308 of preparing the femoral canal for insertion of a vent tube in a vent path through an anterior portion of the femur (e.g. via the greater trochanter), into the femoral canal and along an anterior cortical wall of the femoral canal to a position distal of a distal end of the prosthetic femoral stem component, when inserted in the femoral canal. The method also comprises a step 310 of inserting a vent tube into the vent path such that a tip of the vent tube is located in soft cancellous bone in the femoral canal, for use in venting the femoral canal during subsequent insertion of the prosthetic femoral stem component.

The method 300 may be implemented for either cemented or uncemented stems. However, it is critical that the stem is inserted in the correct position in order to leave adequate space for the vent tube anteriorly of the stem. The stem designs must therefore be flat (blade stems) to be used with an anterior positioned vent tube.

Further details of the method 300 are described below.

FIG. 4 shows a view of the top of a femur 400 from above. The femur comprises a femoral head 402, a femoral neck 404, a greater trochanter 406, a lesser trochanter 408 and a trochanteric fossa 410.

When a posterior approach is performed, the external rotators will have been disconnected from their attachments on the femur 400 and upon dislocation of the femoral head 402 from the acetabulum (not shown), the femoral head 402 and femoral neck 404 are easily visible. The lesser trochanter 408 with an attached psoas tendon is visible as is the greater trochanter 406 with attached tendons of gluteus medius and minimus. Disconnection of the external rotators will have disconnected the obturator externus tendon from its insertion in the trochanteric fossa 410. As will be explained below, the trochanteric fossa 410 is a key structure used to obtain correct placement of a total hip replacement stem.

FIG. 5 shows a view down the back (posterior aspect) of the femur 400, showing the longitudinal shaft 500 of the femur. At the bottom (distal end) of the femur 400 a ML reference line is drawn across the backs (posterior aspects) of the lateral femoral condyle 502 and medial femoral condyle 504. It can be seen that the top (proximal) part of the femur 400 is twisted forward (anteriorly) with respect to the ML reference line. This anterior twist is generally referred to as femoral neck anteversion. In practice, the femoral head 402 and femoral neck 404 are not only anteverted, they are also anteriorly displaced.

FIG. 6 shows a transverse section 600 taken through the centre 402c of the femoral head 402 and the centre 404c of the femoral neck 404. The section 600 also passes through the centre 500c of the longitudinal shaft 500 of the femur 400. As illustrated, the ML reference line of FIG. 5 is projected upwardly to form a ML plane and the anteversion angle is determined between the ML plane and the line through the centre 402c of the femoral head 402 and the centre 404c of the femoral neck 404. In the present female subject, the anteversion angle measures approximately 27.2 degrees. However, there are several different definitions of femoral neck anteversion and so, for the same subject, slightly different anteversion angles can be measured according to the definition used.

FIG. 7 shows a side view of the top of the femur 400 after the femoral head 402 and femoral neck 404 have been, respectively, totally and partially resected. This view therefore shows a cut surface 700 through the femoral neck 404.

As indicated in FIG. 7, the cut surface 700 of the femoral neck 404 is greatly encroached upon and minimised postero-laterally by the presence of the trochanteric fossa 410. It is a grave mistake for a novice surgeon to attempt to insert a total hip replacement stem down the cut surface 700 of the femoral neck 404 as this will result in the femoral stem being tilted in the femoral canal and will risk the femoral stem being driven through the posterior wall of the femur 400. Moreover, such tilting of the femoral stem will result in the prosthetic femoral head centre being posteriorly displaced (e.g. by about 7 mm) compared to the natural femoral head centre. This posterior displacement of the prosthetic femoral head will cause impingement, prosthetic dislocation and lack of flexion power due to mechanical disadvantage of the psoas tendon and other flexor muscles.

Consequently, it is important that the hard cortical bone of the trochanteric fossa 410 be partially removed and reshaped, for example, with a powered burr or a small reciprocating saw blade, as shown in FIG. 8. The excavated trochanteric fossa 800 allows straight line access to the femoral canal for instruments such as curettes, reamers, rasps and broaches, to enable the femoral stem component to be positioned in exactly the correct three-dimensional alignment.

FIG. 9 shows a transverse section 900 taken through the excavated trochanteric fossa 800. This shows the greater trochanter 406 on the lateral side, opposite to the medial femoral neck 906. A flat (blade-shaped) prosthetic femoral stem component 902 is correctly positioned in the femoral canal such that the proximal end of the prosthetic femoral stem component 902 is adjacent to a posterior cortical wall 908 of the femoral canal 910, medially of the excavated trochanteric fossa 800. In this position there is plenty of space anteriorly for a vent hole 904 in which to accommodate a vent tube (not shown).

FIG. 10 shows a transverse section 1000 taken through the femur 400 at a position half-way down the prosthetic femoral stem component 902. This shows that there is still space anteriorly for the vent hole 904. In addition, there is now space posteriorly of the prosthetic femoral stem component 902 for a vent hole but, as shown in FIG. 9, there is not sufficient space posteriorly of the prosthetic femoral stem component 902 higher up the femur 400.

FIG. 11 shows a setup 1100 for venting a distal femoral canal during a procedure for inserting cement 1102 for a cemented prosthetic femoral stem component.

In this case, the femoral canal 910 is prepared for insertion of a PMMA vent tube 1104 using, for example, a twist drill on power (not shown) to create a vent path 1106 through an anterior portion of the greater trochanter 406, into the femoral canal 910 and along an anterior cortical wall 1108 of the femoral canal 910 to a position 1110 distal of a distal end of the prosthetic femoral stem component, when inserted in the femoral canal 910. Note, various slices through the femoral canal 910 are illustrated in FIG. 11 to show the relative positions of the vent path 1106 and cement 1102 with respect to the anterior cortical wall 1108 and the posterior cortical wall 1112. In addition, the femoral canal 910 is prepared, for example, by use of vented reamers, rasps and broaches (not shown) for insertion of a prosthetic femoral stem component such that a proximal end of the prosthetic femoral stem component is adjacent to a posterior cortical wall 1112 of the femoral canal 910, medially of the excavated trochanteric fossa 800, as shown in FIG. 9.

When a cemented stem is used, the method comprises inserting a cement restrictor 1120 into the femoral canal 910, distal of the prosthetic femoral stem component. The cement restrictor 1120 has a central threaded hole (not shown) in its upper part for attachment of a long metal cement restrictor inserter (not shown) which will extend out of the prepared upper femur, when in use. The inserter has measurement marks so the cement restrictor 1120 can be inserted to exactly the correct depth in the femoral canal 910. The PMMA vent tube 1104 is loaded in an anterior vent aperture in the cement restrictor 1120 before it is inserted into the femoral canal 910. The vent tube 1104 has a non-fenestrated portion located above the cement restrictor 1120. The distal portion of the vent tube 1104, which is provided with side holes 1114 is located below the cement restrictor 1120. As the cement restrictor 1120 gains a firm fix in the tapering femoral canal 910, the squeeze on the cement restrictor 1120 tightens the fix of the vent tube 1104 in the cement restrictor 1120.

The cement restrictor 1120 comprises the vent aperture for receipt of the vent tube 1104 there-through. The vent aperture may be provided towards an anterior edge of the cement restrictor 1120. The cement restrictor 1120 comprises a stem locator, to aid location of the tip of the prosthetic femoral stem component. The stem locator comprises an indent configured for receipt of the tip of the prosthetic femoral stem component. The stem locator is provided towards a center of the cement restrictor 1120. The cement restrictor 1120 also comprises a plurality of peripheral fins configured to flex on contact with the internal cortical wall 1112 for a snug fit therewith.

The method comprises venting the femur, by operation of a suction device 1202 attached to the PMMA vent tube 1104, whilst injecting liquid cement 1102 with a cement gun into a prepared cavity of the femoral canal 910, above the cement restrictor 1120, prior to insertion of the prosthetic femoral stem component into the cement-filled cavity. As shown in FIG. 11, the suction device 1202 is connected to the vent tube 1104 via a flexible extension tube 1204. In this case, a clear plastic extension tube 1204 is push-fitted onto a PMMA vent tube 1104 and the suction device 1202 is push-fitted into an expanded distal end of the extension tube 1204.

In this case, the vent tube 1104 is a PMMA tube having a plurality of side holes 1114 located at the distal end 1110, for venting the femur distally of the cement restrictor 1120. Attempted use of a polished metal vent tube 1104 with a cemented stem is a risky business. It may not be possible to extract the polished metal vent tube 1104 when the cement has set and then there is a risk of frictional movement and corrosion between the stem and the polished metal vent tube 1104. However, if the surgeon is lucky and skilful and does manage to extract polished metal vent tube 1104 from the cement then the vent path needs to be blocked at the level of the cement restrictor 1120 and the vent path filled with low viscosity cement. As stated elsewhere a polished metal vent tube 1104 is best used with a uncemented flat stem. A PMMA vent tube is ideally used with a cemented flat stem and left in place, blocked at the level of the cement restrictor 1120 and filled full of low viscosity cement.

FIG. 12 shows a setup 1200 for venting a distal femoral canal during a procedure for inserting a cemented prosthetic femoral stem component 1210 into the cement 1102 of FIG. 11. The setup 1200 is similar to that of FIG. 11 but the prosthetic femoral stem component 1210 is fitted with a centraliser 1212 on its distal tip, for location in the stem locator of the cement restrictor 1120. Known art teaches that a PMMA centralizer also provides an air gap at the tip of the stem to allow micro subsidizing of the femoral component within the cement mantle thus providing better fixation of the stem in the cement.

The method comprises venting the femur, by operation of the suction device 1202 attached to the vent tube 1104, whilst conducting pressurization of cement by dedicated instruments and inserting the prosthetic femoral stem component 1210 into the cement 1102 in the prepared cavity of the femoral canal 910.

A procedure for extracting the vent tube from the vent path may be employed, for example, when a stainless-steel vent tube is used and the vent tube requires removal after the prosthetic femoral stem component has been inserted. Such a removable vent tube may be particularly useful for uncemented stem procedures.

The optimum alternative would be to use a PMMA vent tube 1104, for example, with cemented stems, so that the vent tube may remain in situ after the stem has been inserted. In this case, the PMMA vent tube 1104 may be cut level with a top surface of the femur 400, typically at the anterior greater trochanter 406, and then the vent tube 1104 filled with cement 1102 down to the cement restrictor 1120 (as will be described below).

The vent tube 1104 may have a circular, oval or D-shaped cross-section. As an anterior face of a flat (blade-shaped) stem is generally flat and the inner cortical wall of the femur 400 is generally circular, a D-shaped vent-tube 1104 would fit well if orientated such that a curved portion of the vent tube 1104 is located along the anterior cortex wall 1108 of the femoral canal 910, and a flat portion of the vent tube 1104 is arranged to face the prosthetic femoral stem component 1210 in the femoral canal 910.

Circular-section vent tubes 1104 may have a 6 mm outer diameter and a 4 mm diameter bore. A vent tube 1104 with a 5 mm outer diameter and a 3 mm diameter bore would also work fine. A 4 mm outer diameter vent tube 1104 having an inner bore diameter of 2 mm would also work fine. However, smaller diameter vent tubes 1104 would be more prone to blockage by small fragments of reamed cancellous bone and unblocking would require the surgeon to pass a wire down the inside of the vent tube 1104.

The appropriate choice of diameter and shape of vent tube 1104 will be governed by the size of the patient and the size of the femoral canal 910 and also the size and shape of the femoral component. A range of prosthetic femoral component stems typically has 12 sizes and a smaller component is selected for a smaller size of femur 400.

In addition, there are different types of upper femora and these are known as Dorr A, Dorr B and Dorr C. A Dorr A femur, having a thick cortical wall and a narrow femoral canal, is typically found in young men and presents a challenge to insert a cemented stem plus an anterior vent tube. However, the femoral bone quality is excellent in Dorr A femora and many surgeons would select an uncemented femoral stem component. Having said that, there are some surgeons, who never use an uncemented stem and venting of such a femur would require the use of a vent tube 1104 inserted from a distal approach as described below. Dorr B femora, having a mid-thickness cortical wall and a mid-thickness femoral canal, could be treated with either a cemented or an uncemented stem according to surgeon preference. A Dorr C femur, having a thin cortical wall and a wide femoral canal, are typically treated with a cemented stem femoral component and, as the femoral canal is wide, there is no difficulty inserting an anterior vent tube.

As flexible plastic wound drains are available in operating rooms, some surgeons might think that a further alternative would be to use a flexible plastic vent tube 1104. However, in this case, a potential problem may occur if the vent tube 1104 gets stuck and fractures on attempted extraction, following a cemented or uncemented total hip arthroplasty procedure. If a piece of plastic from the vent tube 1104 is left in the patient following surgery, it may result in litigation.

In summary, a removable stainless steel (or other metal) vent tube 1104 is ideally used in flat section (blade) uncemented stem hip arthroplasty in Dorr B and C femora.

An implantable PMMA vent tube 1104 is ideally used with flat cemented stem hip arthroplasty in Dorr B and Dorr C femora and use of a D-shaped section PMMA vent tube 1104 may be advantageous with smaller femoral canals seen in some patients with a Dorr B type femur.

A vent tube 1104 inserted into the femoral canal 910 from a distal approach (as described below) is ideally used in Dorr A femora when the surgeon elects to use a cemented stem. However, a distal approach vent tube 1104 can be used with cemented or uncemented stems, round or flat section stems in Dorr A, B or C type femora, if the surgeon prefers a distal vent.

FIG. 13A shows a tool 1600 for inserting a deformable plug 1602 into a PMMA vent tube 1104, optimally at the position of the cement restrictor 1120, with the deformable plug 1602 in a compressed configuration for insertion.

FIG. 13B shows the tool 1600 of FIG. 13A, with the deformable plug 1602 in an expanded configuration for blockage in the PMMA vent tube 1104, or in the vent aperture of the cement restrictor 1120.

The tool 1600 comprises a handle 1604, having a trigger 1606 and a shaft 1608 including an inner rod 1610. When the trigger 1606 is compressed, as shown in FIG. 13A, the inner rod 1610 is pushed out of the end of the shaft 1608, which deforms the deformable plug 1602 into a radially compressed configuration for insertion. When the trigger 1606 is released, as shown in FIG. 13B, the inner rod 1610 is retracted into the end of the shaft 1608, which allows the deformable plug 1602 to expand radially for blocking a passage. An outer surface of the shaft 1608 may comprise measurement markings to aid with the correct location of the deformable plug 1602 in the PMMA vent tube 1104.

FIG. 14 shows injection of cement 1102 into the PMMA vent tube 1104. Thus, the method may comprise injecting liquid cement 1102 into the PMMA vent tube 1104, above the deformable plug 1602, to fill the PMMA vent tube 1104 between the cement restrictor 1120 and an entrance into the femur 400. A syringe 1800 and cannula 1802 may be used to fill the PMMA vent tube 1104 from a distal end up to a proximal end as the cannula 1802 is gradually withdrawn. The purpose of the flexible plug is to prevent liquid cement from being injected into the distal femur.

FIG. 15 shows a metal vent tube 1104 inserted into a prepared femoral canal 910 following preparation of the femoral canal 910 for an uncemented prosthetic femoral stem component 2100. The femoral canal 910 may be prepared for the vent tube 1104 or stem 2100 by way of a vented reamer, rasp or broach (not shown). As described previously, an extension tube 1204 is attached to a proximal end of the vent tube 1104 and a suction device 1202 is attached to a proximal end of the extension tube 1204. The vent tube 1104 extends to a distal region of the femur 400 and is configured to vent the distal femur during insertion of the uncemented prosthetic femoral stem component 2100.

FIG. 16 shows suction being applied to the vent tube 1104 during insertion of the uncemented prosthetic femoral stem component 2100.

FIG. 17 shows a pole 1302 inserted into the vent tube 1104 for extraction of the vent tube 1104. The pole 1302 is formed from a thick wire or rod configured to match an internal bore diameter of the vent tube 1104.

FIG. 18 shows pliers 1400 being used to grip the vent tube 1104 and pole 1302 therein, for removal of the pole 1302 and vent tube 1104 from the femur 400.

FIG. 19 shows the open vent path 1106 after removal of the vent tube 1104. In this case, if an uncemented stem is used, small cancellous bone chips may be packed into the top of the vent path 1106 after vent tube 1104 removal. The bone chips will fuse into strong bone to permanently block the vent path 1106.

FIG. 20 shows a vent tube 1104 having a circular cross-section, in position during a cemented stem total hip arthroplasty. This figure shows various transverse sections taken along the femoral canal 910 showing the relative positions of the vent tube 1104 and the prosthetic femoral stem component 1210. Thus, it can be seen that at a proximal end of the prosthetic femoral stem component 1210, the vent tube 1104 is displaced anteriorly of the prosthetic femoral stem component 1210, which is located close to the proximal cortical wall 1112. Further down the femoral canal 910, the prosthetic femoral stem component 1210 is located more towards the centre of the femoral canal 910, while the vent tube 1104 remains adjacent to the anterior cortical wall 1108.

Ideally a PMMA vent tube is designed to be used with cemented stems and left in-situ. After curing of cement, the PMMA vent tube is cut off at the top of the anterior trochanter, then blocked at the level of the cement restrictor, and then filled with low viscosity cement.

For an uncemented flat stem, ideally a stainless-steel vent tube is used and after the stem has been inserted, the vent tube is pulled out and the top of the hole in the bone is bone grafted.

All the other iterations described and drawn are less preferred when compared to the two options above. For example, a soft plastic vent tube could be used with an uncemented stem but it risks breaking on attempted removal after the uncemented stem is inserted. A stainless-steel vent tube could be used with a cemented flat stem but there is a risk that the stainless-steel tube could not be removed after insertion of the cemented stem. This is undesirable as leaving the stainless-steel tube in-situ could cause fretting corrosion against the metal of the cemented stem. A round or oval cross-sectional PMMA vent tube could be used in cemented flat stems but they are not as good as a D-section tube as this creates the biggest gap between the stem and the cortex of the femur. A PMMA vent tube for use with cemented stems is ideal as chemically the material of the vent tube and the wet cement inserted for stem fixation are the same and wet cement is likely to chemically bond to the outer surface of the vent tube. Holes in the proximal part of the vent tube are an option but they increase complexity for a surgeon and if the holes end up in the canal of the femur rather than the cancellous bone at the top of the femur then wet cement can be sucked into the holes and will block the vent path.

FIG. 21 shows a D-shaped PMMA vent tube 1104, which has been inserted into the femur to remain in situ after insertion of a flat cemented prosthetic femoral stem component 2100. This figure also shows various transverse sections taken along the femoral canal 910 showing the relative positions of the vent tube 1104 and the prosthetic femoral stem component 2100. Thus, it can be seen that at a proximal end of the prosthetic femoral stem component 2100, the vent tube 1104 is displaced anteriorly of the prosthetic femoral stem component 2100, which is located close to the posterior cortical wall 1112. Further down the femoral canal 910, the prosthetic femoral stem component 2100 is located more towards the centre of the femoral canal 910, while the curved surface of the vent tube 1104 remains adjacent to the anterior cortical wall 1108. Moreover, the flat surface of the D-shaped vent tube 1104 allows more room for the prosthetic femoral stem component 2100 in the narrow portions of the femoral canal 910.

FIG. 22 illustrates a vertical plane 2700 through a femur 400 and cemented prosthetic femoral stem component 1210 to illustrate another design of vent tube 2702.

FIG. 23 shows a vertical section corresponding to the plane 2700 of FIG. 22 and with an extension tube 1204 and suction device 1202 attached to the vent tube 2702. FIG. 23 shows that the vent tube 2702 has the same distal side holes 1114, distal to the cement restrictor 1120, as shown previously. However, the vent tube 2702 also has proximal side holes 2704 located in the soft cancellous bone of the greater trochanter. This variety of vent tube 2702 with proximal side holes 2704 does require more surgeon skill and comes with risk of accidental blockage of the vent tube 2702.

It is believed that the distal femur is the main source of systemic embolisation during total hip arthroplasty. It is known that despite inserting a cement restrictor 1120, the pressure in the distal femur during cemented stem arthroplasty reaches 1400 mm Hg (186651.31579 Pascals). It is thought that the thin layer of cancellous bone on the inner cortical bone bounding the femoral canal 910 prevents a perfect seal of the cement restrictor 1120 which allows pressure generated in the proximal femur to be transmitted to the distal femur. This causes embolisation into veins allowing fat and bone marrow to travel to the right side of the heart and get stuck in the pulmonary vascular tree.

There is some cancellous bone left in the proximal femur after preparation for total hip arthroplasty and the proximal side holes 2704 are provided in the vent tube 2702 to address any high pressure being experienced by this remaining proximal cancellous bone thereby reducing the risk of embolisation. As above there are downsides. In addition, it is very easy for a surgeon to decompress the proximal femur by a small metal vent tube inserted into the lesser trochanter (as is done with Hip Resurfacing) or by a vent tube inserted into the cancellous bone of the greater trochanter.

FIG. 24 illustrates a transverse plane AA through the femur 400 and cemented prosthetic femoral stem component 1210 of FIGS. 22 and 23.

FIG. 25 shows a transverse section corresponding to the plane AA of FIG. 24, illustrating the proximal side holes 2704 in the proximal portion of the vent tube 2702. As explained above, the vent tube 2702 is configured with proximal side holes 2704 to keep pressure low in the cancellous bone of the anterior, lateral and posterior greater trochanter region.

An alternative to using the vent tube 2702 with both distal and proximal side holes 1114, 2704 is to only use the vent tube 1104 with distal vent holes 1114 but to additionally use a vent cannula (not shown) inserted directly into the greater trochanter cancellous bone or into the lesser trochanter (as above).

### Distal Approach

FIG. 26 shows a flow diagram of a method 3100 of preparing a femur for hemi arthroplasty or total hip replacement using a distal approach. This method may be used with circular-section or flat-section stems, which are either cemented or uncemented.

In the method 3100 of preparing a femur for hemi arthroplasty or total hip replacement, the femur 400 comprises a lateral femoral condyle, a femoral canal and a proximal end. The method 3100 comprises a step 3102 of cutting into a non-distal portion of the lateral femoral condyle to form a part of a vent path and a step 3104 of introducing a cutting tool into the part of the vent path, the cutting tool comprising a hollow tube having a cutting blade mounted at a tip thereof. The method further comprises a step 3106 of inserting a rod into the hollow tube of the cutting tool, the rod having a substantially straight shaft and a curved tip configured to guide the cutting blade in a curved path. The method further comprises a step 3108 of manoeuvring the cutting tool, in conjunction with the inserted rod, in order to extend the vent path, along the curved path defined by the curved tip, until the cutting blade is substantially aligned with a longitudinal axis of the femoral canal. The method further comprises a step 3110 of removing the rod and advancing the cutting tool along the femoral canal, to extend the vent path in a direction towards the proximal end of the femur. The method further comprises a step 3112 of removing the cutting tool to expose the vent path; and a step 3114 of inserting a vent tube into the vent path such that a tip of the vent tube is located in soft cancellous bone in the femoral canal, for use in venting the femur during subsequent hemi arthroplasty or total hip replacement.

Further details of the method 3100 are described below.

FIG. 27 shows a CT scan of a femur 400 of an elderly lady, with vent cannulas 3200, 3202 inserted in both the proximal and distal ends of the femur 400.

The proximal femur is typically vented through the lesser trochanter during hip resurfacing and the cancellous bone here is sparse. As such, using a proximal vent cannula 3200 is fully effective in preventing embolisation into the right heart and lungs during hip resurfacing.

By contrast, the distal femur contains dense cancellous bone and the applicant's experience of venting here using a vent cannula 3202 during cemented stem THA was totally ineffective at preventing embolisation into the right heart.

The applicant therefore proposes to use the distal approach of the method 3100 in order to vent the mid femoral canal effectively by passing a vent tube through the dense cancellous bone of the distal femur and into more sparse cancellous bone in the mid femur, which is likely more suitable for successful venting.

FIG. 28 shows an initial cut being made into the lateral femoral condyle 502 using a cutting tool 3300 to form a part of a vent path. The cutting tool 3300 comprises a hollow tube 3302 having a cutting blade 3304 mounted at a tip thereof. The hollow tube 3302 may be formed of a resilient material configured to revert to a rest position which is substantially in the form of a right circular cylinder.

The cutting tool 3300 is provided with a handle 3306 extending at an angle to the longitudinal axis of the hollow tube 3302, when in the rest position. The handle 3306 is provided with a blade 3308 for making the initial cut (e.g. hole) into the lateral femoral condyle 502.

In use, an initial cut is first made from a side of the lateral femoral condyle 502 with the blade 3308. The handle 3306 is then rotated such that the blade 3308 enters the lateral femoral condyle 502 through the thin cortex of the lateral femoral condyle 502. After penetrating the bone by approximately 1 cm or more, the blade 3308 is removed from the lateral femoral condyle 502.

FIG. 29 shows the cutting blade 3304 of the cutting tool 3300 being inserted into the initial cut in the lateral femoral condyle 502. In addition, a rod 3400 is inserted into the hollow tube 3302. The rod 3400 has a substantially straight shaft 3402 and a curved tip 3404 configured to guide the cutting blade 3304 in a curved path. The rod 3400 is formed of metal and configured to substantially hold its shape when in use.

The rod 3400 is provided to assist the surgeon in turning the corner when the cutter blade 3304 is rotated.

FIG. 30 shows the cutting tool 3300 and rod 3400 after the vent path has been extended in a curved path. From this view, it can be seen that the handle 3306 is angled such that the handle 3306 is substantially aligned with the longitudinal axis of the femoral canal 910, when the cutting blade 3304 is substantially aligned with a longitudinal axis of the femoral canal 910, after the cutting blade 3304 has followed the curved path defined by the curved tip 3404 of the rod 3400. In other words, the angle of the handle 3306 with respect to the hollow tube 3302 is related to (e.g. supplementary to) an angle of the curved tip 3404 of the rod 3400 with respect to the shaft 3402 of the rod 3400.

The method 3100 may comprise imaging the femur 400 to monitor and/or verify the position of the cutting blade 3304 in order to ensure accurate creation of the distal vent path. The imaging may comprise digital fluoroscopy or computerized navigation.

FIG. 31 shows a view similar to that of FIG. 30 but with the rod 3400 removed.

FIG. 31 shows that the surgeon then rotates the handle to advance the cutter end up the canal of the femur. Again, this can be verified by fluoroscopy.

FIG. 32 shows the cutting tool 3300 advanced further up the femoral canal 910 by rotation of the handle 3306 (e.g. about the longitudinal axis of the hollow tube 3302). The position of the cutting blade 3304 in the femoral canal 910 can be monitored and/or verified by imaging (e.g. fluoroscopy). Once the vent path is as far as desired along the femoral canal 910, the cutting tool 3300 is withdrawn.

As shown in FIG. 33, a flexible guide wire 3800 is then inserted along the vent path and beyond the created vent path into the mid femur 400.

FIG. 34 shows a vent tube 3900 being threaded along the guide wire 3800. The vent tube 3900 comprises side holes 3902 at the end fed up into the mid femur 400.

FIG. 35 shows the vent tube 3900 advanced along the guide wire 3800 such that the side holes 3902 are located in soft cancellous bone in the mid femur 400.

FIG. 36 shows the vent tube 3900 in position after removal of the guide wire 3800.

FIG. 37 shows a suction device 1202 attached to the vent tube 3900 for venting the femur 400.

### Venting the Femoral Shaft of Femur During Hip Arthroplasty

The applicant has over 25 years of experience successfully venting the femur during approximately 5,000 hip resurfacing (HR) operations where the hollow femoral component that caps the femoral head employs cement fixation and also in ultra-short stem (USS) hip replacements which employ uncemented fixation in the neck of the femur and the trumpet shaped femoral head-neck junction and the stem does not enter or rely on fixation in the shaft of the femur. Insertion of both these types of implants increases the pressure in the medullary canal of the shaft of the femur. The medullary canal of long bones is where the body forms blood in bone marrow and other important cells. In the medullary canal of bones is found cancellous bone, cells that form blood in bone marrow, blood and fat. The medullary canal of the femur has supporting cancellous bone. This cancellous bone can easily be fractured by insertion of any instrument or vent tube into either the shaft of the femur or the cancellous bone of the femoral head and neck. This fractured cancellous bone is a constant threat to blocking any vent tubes and the surgeon must be vigilant to unblock vent tubes otherwise the attempt to vent the femur fails.

Any increased pressure in the shaft of the femur causes blood, fat and marrow to be driven into the veins which drain the femoral bone to veins in the leg and this fat and marrow eventually travels up the inferior vena cava in normal blood and into the right atrium and right ventricle of the heart. The applicant's anaesthiologist was expert at performing Trans-oesophageal Echo Cardiography (TOE) and these TOE images were displayed on a large screen in the operating room for all staff to view. Many lessons were learned from real time TOE on hip arthroplasty patients.

During HR operations, if the femoral canal is not vented, embolization of fat and marrow into the right side of the heart occurs.

Insertion of a metal cannula vent through the lesser trochanter into the femoral canal does nothing on its own to stop the above embolization.

Attaching a clear plastic tubing connected to strong surgical suction to the metal vent cannula has a consistent positive effect. When the clear plastic suction tube is attached, flow of blood is seen to trickle up the clear plastic tube. If this flow of blood stops, the vent apparatus is blocked and this blockage must be cleared immediately to restore effective venting.

When the HR is being fitted, the trickle of blood through the clear plastic tubing changes suddenly to high flow of either 1) fat which is seen as white through the tubing wall, 2) marrow which is seen as a dense red colour or 3) high flow of normal blood and eventually 4) a trickle of normal blood as before the HR procedure was started.

The effect of the surgical procedure on massively increasing the pressure in the shaft of the femur is only seen on TOE after the prosthetic femoral head is reduced into the acetabulum. The commonest surgical approaches of posterior approach or antero-lateral approach both rely on twisting the femur into either extreme internal rotation or extreme external rotation to present the dislocated femoral head to the surgeon to be able to perform either HR, USS hip replacement or conventional THA stem replacement, either cemented or uncemented.

Real time TOE shows that no embolization into the heart is seen during insertion of HR, USS or THA. Embolisation into the heart or lack of embolization is only seen on TOE after the twist on the femur and femoral veins has stopped by reducing the prosthetic femoral head into the acetabulum.

This means that the surgeon is unaware of whether the hip arthroplasty he/she is performing is causing massive embolization of fat and marrow by viewing real time TOE. Only after reduction of the prosthetic femoral head into the acetabulum (with or without a prosthetic surface on the acetabulum-as in hemi-arthroplasty undertaken for femoral neck fractures in the elderly) is performed, is the surgeon aware of the huge embolization injury that they have caused.

Intra-operative TOE is not performed routinely at hip arthroplasty. All arthroplasty surgeons know that they are causing massive embolisation at hip arthroplasty. However, at the commonest THA procedure, there is currently no method for preventing massive emblolisation into the right side of the heart with the fat and marrow embloli getting jammed in the pulmonary vascular tree. The applicant presents his failed attempts to vent the femur during conventional THA, mainly cemented stem THA, but also during uncemented THA. Only rarely do THA patients have the early and dangerous fat embolism syndrome. However, elderly people having cemented stem hemi arthroplasty for a fractured neck of femur do get fat embolism syndrome. Most THA patients have no clinical evidence of massive embolization either during operation or post-operatively. Excess death rates are obvious in the years following THA but massive population-based studies are required to demonstrate this excess mortality.

Evidence as to why fat and marrow embolization at the time of THA surgery causes late excess mortality comes from excess mortality from thrombotic pulmonary embolism (TPE). TPE either kills the patient immediately or, in the majority, they survive the initial incident. Late mortality is caused by pulmonary hypertension and right-sided heart failure. It may be that late increased mortality following fat and marrow embolization during THA results in late pulmonary hypertension and right sided heart failure which causes later excess death.

It is therefore an aim of the present disclosure to provide methods of preparing a femur for insertion of a prosthetic femoral stem component, with improved patient outcomes following hip arthroplasty.

### Cemented Stems

It is difficult to keep the distal femoral pressure low during insertion of a cemented stem.

It will be clear that a cemented stem with a vent canal down the length of the stem, on its own, will be completely ineffective at reducing pressure rise in the distal femur as sucking at the top of the vent canal will merely result in sucking semi-liquid cement into the bottom of the vent canal thereby contributing nothing to keep pressure in the mid and distal femur from rising to dangerous levels.

The applicant tried inserting a vent tube into the distal femur, through the lateral femoral condyle, during stemmed THA procedures. To their surprise, this attempted venting did nothing to prevent massive and prolonged embolisation into the right atrium (RA) and right ventricle (RV). They tried inserting two vents and inserting a wide bore vent tube, but massive embolisation still continued. It was not until many years later that they understood the reason for this lack of effectiveness of a distal vent tube. Whole femur computerised tomography (CT) scanning showed that in middle aged and elderly patients the proximal and middle thirds of the femoral canal was only filled by sparse cancellous trabecular bone. However, the distal third of the femoral canal is full of dense cancellous trabecular bone. This dense cancellous trabecular bone blocks successful venting through the lateral femoral condyle. In the proximal femur, strong dense cancellous bone is only found in the femoral head and neck and this may be used to support resurfacing femoral components and ultra-short stem femoral components which are anchored in the femoral head and femoral neck.

The most commonly used cemented stems at the time of writing, particularly in the elderly, are polished tapered stems with the so-called Exeter stem being the market leader. The aim with the Exeter stem is not to get a firm fix in the femoral canal of the femoral stem to the bone cement. Instead, a slip fit only of the stem in the femoral canal cement is desired with a complete cement mantle separating the stem from the bone on the inside of the femur.

The Exeter stem and other polished tapered stems employ a cement restrictor (typically of polyethylene) inserted into the femoral canal below the femoral stem tip. Liquid cement, such as poly methyl methacrylate (PMMA), is then injected into the washed and dried bone of the femoral canal and then pressurised until the cement becomes doughy. Next, the femoral stem component with a factory cured PMMA centraliser (also known as a subsidiser) attached to its tip is inserted into the cement to a desired depth to result in a correct leg length. The femoral cement is then allowed to cure, typically over 10 to 14 minutes.

Polished tapered stems work by allowing slight distal movement of the stem within the cement mantle. In order to obtain this, it is vital that the stem is not allowed to ‘stand on its end’. The subsidiser has a deliberate air gap immediately distal to the stem tip, which could otherwise hamper distal movement and jamming of the stem within the cement mantle. A correctly designed and inserted polished tapered stem, by being free to slightly slip within the cement mantle, results in radially directed forces giving compression at the bone and cement interface. Bone likes compression and acts positively. On the other hand, a stem which is locked to the cement mantle will transmit shear forces to the bone and cement interface and this will contribute to stem loosening.

It is therefore an aim of the present disclosure to provide an adapted method of cementing a prosthetic femoral stem component, into a femur whilst enabling venting of the femoral canal. To achieve this aim, modification must be made to the traditional cement restrictor and the centraliser (e.g. subsidiser) in addition to including a vent hole down the femoral stem.

FIG. 38 shows a flow diagram illustrating a method 4000 of cementing a prosthetic femoral stem component into a femur 5400, in accordance with the present disclosure.

The method 4000 comprises a first step 4002 of inserting a cement restrictor 4100, having a bore there-through, into a femoral canal at a position determined to be below a position of the distal tip of the prosthetic femoral stem component, when located therein, as illustrated in FIG. 39.

The cement restrictor 4100 comprises a proximal plug portion 4102 and a perforated polyethylene tube 4104 extending distally therefrom, wherein the bore extends through the plug portion 4102 and the perforated tube 4104.

The method 4000 further comprises a step 4004 of inserting an extension tube 4200 into the bore of the cement restrictor 4100, as illustrated in FIG. 40.

The extension tube 4200 is formed of PMMA and comprises an enlarged portion 4202 at the proximal end, for accommodating (directly or indirectly) an end of the suction device 1202 and a distal tip 4310 of the of the prosthetic femoral stem component 4300 (shown in FIGS. 48A-C), preferably with an air gap distal to the femoral component tip 4310 so as to also serve as subsidizer. The extension tube 4200 is substantially straight and has an internal diameter of 4 mm whereas the enlarged portion 4202 has a diameter of at least 6 mm. The extension tube 4200 may have an outer diameter of 6 mm along all but the enlarged portion 4202.

The method 4000 further comprises attaching a suction device 1202 through a proximal end of the extension tube 4200, as illustrated in FIG. 41, and, in a step 4006, operating the suction device 1202 whilst injecting liquid cement 4400 into the femoral canal around the extension tube 4200, as illustrated in FIG. 42.

The liquid cement 4400 may be injected into the canal of the femur 5400 using a cement gun (not shown) and then pressurized with a flexible plastic bulb (not shown) until the cement 4400 is doughy after roughly 4 minutes of mixing.

The method 4000 further comprises a step 4008 of removing the suction device 1202 from the proximal end of the extension tube 4200 and a step 4010 of inserting the distal tip 4310 of the prosthetic femoral stem component 4300 into the proximal end of the extension tube 4200, as illustrated in FIG. 43.

The prosthetic femoral stem component 4300 comprises a substantially longitudinal stem portion 4302; a neck portion 4304 having a longitudinal axis intersecting the longitudinal stem portion 4302; and a vent hole 4306 extending through the longitudinal stem portion 4302 from a proximal surface 4308 to the distal tip 4310. The vent hole 4306 comprises an enlarged portion 4312 adjacent to the proximal surface 4308, for accommodating an end of the suction device 1202 during use. The enlarged portion 4312 has a threaded bore for threaded connection to other components as will be described below. The vent hole 4306 has a tapered portion 4314 connecting the enlarged portion 4312 to a remainder of the vent hole 4306. The vent hole 4306 is substantially straight and has a diameter of 4 mm. The prosthetic femoral stem component 4300 is made of metal.

The method 4000 further comprises a step 4012 of inserting the suction device 1202 into the enlarged portion 4312 of the vent hole 4306 of the prosthetic femoral stem component 4300; and a step 4014 of operating the suction device 1202 whilst inserting the prosthetic femoral stem component 4300 into the femoral canal to a desired depth to give the correct leg length, and waiting for the cement 4400 to cure to fix the prosthetic femoral stem component 4300 in the femoral canal, as illustrated in FIG. 44.

Operation of the suction device 1202, as described, transmits suction to the mid and distal femur preventing a dangerous rise in pressure.

The air gap created below the distal tip 4310 of the prosthetic femoral stem component 4300 allows micro-distal migration of the stem portion 4302 within the cement mantle.

Notably, the perforated tube 4104 of the cement restrictor 4100 is configured to accommodate the extension tube 4200, when the prosthetic femoral stem component 4300 is fixed in the femoral canal, as illustrated in FIG. 44.

FIG. 45 shows a view similar to that of FIG. 49 but with the suction device 1202 removed and a stopper 4350 inserted into the vent hole 4306 of the prosthetic femoral stem component 4300, in accordance with the present disclosure. The stopper 4350 shown in FIG. 45 is a hex socket screw-in plug, which allows for a secure and tamper-resistant fastening method.

### Cemented Stems for Distal Migration

An example of a known Exeter stem 6000 for cement fixation is shown in FIG. 46. This stem 6000 comprises a neck portion 6010 and a stem portion 6012. The stem portion 6012 has a front to back dimension that is less than the anterior to posterior dimension of the femur. The stem portion 6010 is also wider medio-laterally (side to side) than in an anterior to posterior (front to back) direction and therefore it is known as a blade stem. The Exeter stem 6000 is flat on the anterior and posterior surfaces of the stem portion 6012 throughout its length. The Exeter stem 6000 has a small radius curve 6014 at the upper lateral aspect of the stem portion 6012.

The inventor has observed that for such Exeter stems 6000, follow up x-rays 5 years post-operatively, reveal that nearly all patients have experienced migration of the stem 6000, which can be seen by a gap in the cement mantle at the top lateral side. As shown in FIG. 46, when the Exeter stem 6000 distally migrates (i.e. when the distal tip migrates distally from location 6004a to 6004b), the small radius curve 6014 of the stem 6000 parts from the surrounding cement mantle resulting in a tapering curved gap, illustrated between points 6002a and 60002b, with no fixation to bone in that area. Unloaded bone undergoes osteopenia with weakening of this region of the femur bone.

There is accompanying medial translation of the upper portion of the stem 6000, as shown in FIG. 46, but that is not normally noticeable by surgeons. For example, point 6006a on the upper medial aspect is translated to point 6006b. In the case of the Exeter stem 6000, the lower medial aspect is migrated along the straight taper of the blade stem portion 6012 from point 6008a to 6008b.

In the translated position, the patients have no pain and have good function. Instead of the bone at the upper medial side fracturing, the bone remodels to accommodate the medial translation of the stem 6000 from point 6006a to point 6006b. However, a problem can arise, particularly in elderly patients with weak bone-if they fall at any postoperative time point, then their femur can fracture. In which case, an acute forced distal migration (e. g, of 5 mm) of the stem 6000 in the cement is accompanied by a similar acute medial translation of the stem 6000, which can fracture the upper medial femur in the region 6006b. The medial aspect of the femur is already weakened by resection of the femoral head and at least half of the femoral neck during THA. In addition, there is little soft cancellous bone to provide a buffer between the medial side of the Exeter stem 6000 and the hard cortical bone of the femur. This risky situation can lead to peri-prosthetic femoral fracture particularly in elderly people if they have a fall. However, this fracture is generally not confined to the upper medial femur. The spreading of the fracture may depend on the loading regimen at the fall. If there is a twisting element accompanying the fall, then the fracture can propagate far down the femur, in some circumstances well below the tip of the stem 6000. The variety of different types of peri-prosthetic fractures are documented in the Vancouver classification. The different types of fractures need different operative approaches, different length and types of internal fixation, with some, in addition, needing revision of the original stemmed implant.

Unfortunately, such peri-prosthetic fractures are not rare. The Australian joint replacement register has reported peri-prosthetic fractures after the un-cemented Corail stem. In small and medium sized stems, out of 51,760 recipients, 3.1% had a peri-prosthetic fracture. Out of 7,758 recipients of a large Corail stem, 3.9% had a peri-prosthetic fracture. Having a peri-prosthetic fracture as a result of a fall is very bad news for elderly patients and surgery and resulting slow mobilisation from such a fall carries a high mortality rate.

The applicant has therefore designed a prosthetic femoral stem component 7000, as shown in FIG. 47, which does not result in the same medial translation as described above. For clarity no vent path is shown in FIG. 47, although it will be understood that a vent path 8000, similar to those described above, would be provided as shown in FIGS. 48A-C.

The prosthetic femoral stem component 7000 is specifically designed to reduce dislocation and peri-prosthetic femoral fracture. The stem 7000 comprises a neck portion 7010 and a stem portion 7012. The stem portion 7012 has a round cross-section distally and gradually transitions into an oval cross-section about one third of the distance from the tip 7004. The oval cross-section is wider medio-laterally than in an anterior to posterior direction. The anterior to posterior dimension of the stem portion 7012 is designed to be similar to the anterior to posterior dimension of the femur in which it is implanted.

As best illustrated in FIG. 48B, on the medial side of the stem portion 7012 there is a single outwardly concave radius medial curve 7500 that generally follows the curvature of the inner medial cortical wall of the femur. As such, the length and/or curvature of the medial curve 7500 will alter with different sizes of stem 7000. The medial curve 7500 should have a single radius of curvature from the junction 7006 of the neck 7010 to the distal third of the stem 7000. Approximately two thirds of the way down the stem portion 7012 towards the distal tip 7004, the medial curve 7500 transitions into a round cross-sectional cone portion 7014 of the stem portion 7012, which extends to the tip 7004. The cone portion 7014 is angled perfectly in harmony with the direction of migration of the stem 7000 which is induced by the medial curve 7500. Referring back to FIG. 47, the medial profile of the stem 7000 ensures that distal migration of the tip from point 7004a to point 7004b is not accompanied by medial translation of the stem 7000. Instead as the junction 7006 between the neck portion 7010 and the stem portion 7012 migrates from point 7006a to point 7006b, the medial curve 7500 ensures that the stem 7000 migrates along the radius of the medial curve 7500, for example, point 7008a is migrated to point 7008b without any medial translation.

Referring back to FIG. 48B, on the lateral side of the stem 7000, there are three parts. The distal third is formed by the cone portion 7014. A central curved lateral portion 7504 of the stem portion 7012 comprises a large outwardly convex radius of curvature (much larger than that of the medial curve 7500) forming a lateral border which does not follow the inner lateral cortical wall of the femur. The central curved lateral portion 7504 laterally cannot continue to the top proximal end of the stem portion 7012, otherwise the lateral top of the stem portion 7012 would impinge on the inner aspect of the greater trochanter and would fracture the greater trochanter. Accordingly, a top curved lateral portion 7506 is provided at the lateral top of the stem portion 7012, having a much smaller radius of curvature than the medial curve 7500 and the central curved lateral portion 7504. This means that the lateral top of the stem portion 7012 will not impinge on the greater trochanter. The top curved lateral portion 7506 extends to a generally transverse proximal surface 7508 of the stem portion 7012. Referring back to FIG. 47, distal migration of the stem 7000, results in the junction 7002 between the top curved lateral portion 7506 and the transverse proximal surface 7508 moving both distally and laterally from point 7002a to point 7002b. Similarly, the central curved lateral portion 7504 is laterally translated along its length.

As can be seen, distal migration of the stem 7000, as shown in FIG. 47, will not result in the stem 7000 parting company with the cement mantle 9000 of FIG. 48C, thus maintaining fixation and bone loading. Thus, the stem 7000 is specifically designed to migrate without translation around the medial side and instead specifically translates solely on the lateral (safer) side.

As described above, the presence of the vent path 8000 allows for suction to be applied using any of the techniques described in this disclosure, in order to reduce the risk of embolisation.

Notably, the stem 7000 may be cemented in the femur using any of the techniques described above. As such, the extension tube 4200 described above may be employed when implanting the stem 7000. Once the stem 7000 is implanted in the femur, a stopper 8002 may be inserted in the enlarged end of the vent path 8000, as shown in FIGS. 48B and 48C.

Peri-prosthetic fractures of traditional stems are a major problem with both uncemented stems and straight polished collarless stems like the Exeter stem described above. The fact is that with a polished tapered stem migrating into the bone cement, a translation has to occur somewhere. The inventor considers that all of the translation would preferentially be in a lateral direction where a) the femoral bone is not weakened so much by resection of the femoral head and neck, and b) there is much more cancellous bone on the lateral side of the femur to serve as a buffer, particularly in the greater trochanter region between the lateral side of the metal stem and the cortical bone of the femur. The applicant has therefore designed the stem 7000, for cement fixation, with an aim of minimising femoral fracture and considers it likely that with this curved stem design the risk of peri-prosthetic fracture would be reduced.

The stem 7000 may be machined and polished from forgings of high nitrogen stainless steel. The risk area for fracture of such femoral stem components is at the junction of the proximal two thirds of the stem and the distal one third of the stem. At this junction, the cross-section of the stem is preferably circular and the vent path 8000 should be located centrally through the circular cross-section. The vent path 8000 may be created by gun drilling. In which case, drilling may be made from both a distal and a proximal direction. For the size of stem 7000 illustrated, both distal and proximal drilling will be on the same centre line. For other stem sizes it is possible that a slight angulation of the proximal drill hole will be required.

### Uncemented Stems

FIG. 49 illustrates a method of implanting the prosthetic femoral stem component 4300, as described above, into a femur 5400, without using cement. The method comprises preparing the femur 5400 for insertion of the prosthetic femoral stem component 4300 (as is known in the art); inserting a suction device 1202 into the enlarged portion 4312 of the vent hole 4306 of the prosthetic femoral stem component 4300; and operating the suction device 1202 whilst inserting the prosthetic femoral stem component 4300 into the femur 5400.

Once the prosthetic femoral stem component 4300 has been inserted into the femur 5400, the suction device 1202 is removed and the stopper 4350 is screwed into the enlarged portion 4312 of the vent hole 4306, as described previously.

Embodiments of the present disclosure can be employed in hemi-arthroplasty or total hip arthroplasty (THA), to reduce a risk of embolisation and death by effectively venting at least a distal portion of the femur during femoral stem insertion.

The skilled person will understand that in the preceding description and appended claims, positional terms such as ‘above’, ‘below’, ‘along’, ‘side’, etc. are made with reference to conceptual illustrations, such as those shown in the appended drawings. These terms are used for ease of reference but are not intended to be of limiting nature. These terms are therefore to be understood as referring to an object when in an orientation as shown in the accompanying drawings.

Although the disclosure has been described in terms of preferred embodiments as set forth above, it should be understood that these embodiments are illustrative only and that the claims are not limited to those embodiments. Those skilled in the art will be able to make modifications and alternatives in view of the disclosure, which are contemplated as falling within the scope of the appended claims. Each feature disclosed or illustrated in the present specification may be incorporated in any embodiments, whether alone or in any appropriate combination with any other feature disclosed or illustrated herein.

1. A method of preparing a femur for insertion of a prosthetic femoral stem component, the femur comprising a femoral head, a femoral neck, a trochanteric fossa, a greater trochanter and a femoral canal, the method comprising:
amputating the femoral head and a portion of the femoral neck;
excavating the trochanteric fossa;
preparing the femoral canal for insertion of the prosthetic femoral stem component such that a proximal end of the prosthetic femoral stem component is adjacent to a posterior cortical wall of the femoral canal, antero-medially of the excavated trochanteric fossa;
preparing the femoral canal for insertion of a vent tube in a vent path through an anterior portion of the femur, into the femoral canal and along an anterior cortical wall of the femoral canal to a position distal of a distal end of the prosthetic femoral stem component, when inserted in the femoral canal; and
inserting a vent tube into the vent path such that a tip of the vent tube is located in soft cancellous bone in the femoral canal, for use in venting the femoral canal during subsequent insertion of the prosthetic femoral stem component.

2. The method of claim 1 comprising inserting the tip of the vent tube to a position distal of a tip of the prosthetic femoral stem component, when inserted in the femur.

3. The method of claim 1 wherein the step of preparing the femoral canal for insertion of the vent tube comprises creating a substantially straight vent path in the femur; and/or wherein the step of preparing the femoral canal for insertion of the vent tube comprises using one or more vented tools.

4. The method of claim 1 wherein the step of preparing the femoral canal for insertion of the prosthetic femoral stem component comprises using one or more vented tools.

5. The method of claim 1 wherein the step of preparing the femoral canal for insertion of the vent tube is performed after the step of preparing the femoral canal for insertion of the prosthetic femoral stem component.

6. The method of claim 1 further comprising venting the femur, by operation of a suction device attached to the vent tube, whilst inserting the prosthetic femoral stem component.

7. The method of claim 1 further comprising directly or indirectly attaching a suction device to the vent tube.

8. The method of claim 1 further comprising imaging the femur during the step of preparing the femoral canal for insertion of the prosthetic femoral stem component and/or during the step of preparing the femoral canal for insertion of the vent tube.

9. The method of claim 1 further comprising plugging the vent path to block entry of debris therein.

10. The method of claim 1 wherein the vent path passes through an anterior portion of the greater trochanter.

11. A method of preparing a femur for hemi arthroplasty or total hip replacement, the femur comprising a lateral femoral condyle, a femoral canal and a proximal end, the method comprising:
cutting into a non-distal portion of the lateral femoral condyle to form a part of a vent path;
introducing a cutting tool into the part of the vent path, the cutting tool comprising a hollow tube having a cutting blade mounted at a tip thereof;
inserting a rod into the hollow tube of the cutting tool, the rod having a substantially straight shaft and a curved tip configured to guide the cutting blade in a curved path;
manoeuvring the cutting tool, in conjunction with the inserted rod, in order to extend the vent path, along the curved path defined by the curved tip, until the cutting blade is substantially aligned with a longitudinal axis of the femoral canal;
removing the rod and advancing the cutting tool along the femoral canal, to extend the vent path in a direction towards the proximal end of the femur;
removing the cutting tool from the vent path; and
inserting a vent tube into the vent path such that a tip of the vent tube is located in soft cancellous bone in the femoral canal, for use in venting the femur during subsequent hemi arthroplasty or total hip replacement.

12. The method of claim 11 comprising inserting the tip of the vent tube to a position distal of a tip of the prosthetic femoral stem component, when inserted in the femur.

13. The method of claim 11 comprising extending the vent path into a middle region of the femur; and/or further comprising inserting a guide wire into the vent path prior to inserting the vent tube into the vent path.

14. The method of claim 11 further comprising imaging the femur to monitor the position of the cutting tool in order to ensure accurate creation of the vent path.

15. The method of claim 11 further comprising venting the femur, by operation of a suction device attached to the vent tube, whilst preparing the femur for insertion of the prosthetic femoral stem component; and/or further comprising venting the femur, by operation of a suction device attached to the vent tube, whilst inserting the prosthetic femoral stem component.

16. The method of claim 1 wherein a cemented prosthetic femoral stem component is inserted into the femur or an uncemented prosthetic femoral stem component is inserted into the femur.

17. The method of claim 11 wherein a cemented prosthetic femoral stem component is inserted into the femur or an uncemented prosthetic femoral stem component is inserted into the femur.

18. A method of cementing a prosthetic femoral stem component into a femur, wherein the prosthetic femoral stem component comprises: a substantially longitudinal stem portion; a neck portion having a longitudinal axis intersecting the longitudinal stem portion; and a vent hole extending through the longitudinal stem portion from a proximal surface to a distal tip; wherein the vent hole comprises an enlarged portion adjacent to the proximal surface, for accommodating an end of a suction device during use; the method comprising:
inserting a cement restrictor, having a bore there-through, into a femoral canal at a position determined to be below a position of the distal tip of the prosthetic femoral stem component, when located therein;
inserting an extension tube into the bore of the cement restrictor;
operating a suction device through a proximal end of the extension tube whilst injecting liquid cement into the femoral canal around the extension tube;
removing the suction device from the proximal end of the extension tube;
inserting the distal tip of the prosthetic femoral stem component into the proximal end of the extension tube;
inserting the suction device into the enlarged portion of the vent hole of the prosthetic femoral stem component; and
operating the suction device whilst inserting the prosthetic femoral stem component into the femoral canal and waiting for the cement to cure to fix the prosthetic femoral stem component in the femoral canal.

19. The method of claim 18 wherein the extension tube comprises an enlarged portion at the proximal end, for accommodating an end of the suction device and the distal tip of the of the prosthetic femoral stem component; and/or wherein the cement restrictor comprises a proximal plug portion and a perforated tube extending distally therefrom, wherein the bore extends through the plug portion and the perforated tube.

20. A method of implanting an uncemented prosthetic femoral stem component into a femur, wherein the prosthetic femoral stem component comprises: a substantially longitudinal stem portion; a neck portion having a longitudinal axis intersecting the longitudinal stem portion; and a vent hole extending through the longitudinal stem portion from a proximal surface to a distal tip; wherein the vent hole comprises an enlarged portion adjacent to the proximal surface, for accommodating an end of a suction device during use; the method comprising:
preparing the femur for insertion of the prosthetic femoral stem component;
inserting a suction device into the enlarged portion of the vent hole of the prosthetic femoral stem component; and
operating the suction device whilst inserting the prosthetic femoral stem component into the femur.

21. A prosthetic femoral stem comprising: a neck portion and a stem portion; the stem portion having a distal tip and a vent hole through the stem portion from a proximal surface to the distal tip; the stem portion comprising a concavely curved medial surface having a single radius of curvature extending from a junction between the neck portion and the stem portion to a region a third of the distance from the distal tip; and wherein the vent hole is located centrally through the stem portion in said region.
